(19) United States
(12) Patent Application Publication
(10) Pub. No.: US 2009/0042208 A1
(43) Pub. Date: Feb. 12, 2009

(54) Assays for erectile and bladder dysfunction and vascular health

(76) Inventors: Kelvin P. Davies, New York, NY (US); Arnold Melman, Ardsley, NY (US)

Correspondence Address: AMSTER, ROTHSTEIN & EBENSTEIN LLP, 90 PARK AVENUE, NEW YORK, NY, 10016, US

(21) Appl. No.: 12/221,181

(22) Filed: Jul. 31, 2008

Related U.S. Application Data
(60) Provisional application No. 60/962,647, filed on Jul. 31, 2007.

(51) Int. Cl. C12Q 1/68; C12Q 1/02

(52) U.S. Cl. 435/6; 435/29

(57) ABSTRACT

Assays are provided for erectile and bladder dysfunction and vascular health (e.g., cardiovascular disease, hypertension), where the assays detect the expression of one or more of the human Vcsa1 gene family. The assays are also useful for monitoring the efficacy of treatment of these disorders.

### CROSS-REFERENCE TO RELATED APPLICATION

This application claims the benefit of U.S. Provisional Patent Application No. 60/962,647, filed Jul. 31, 2007, the content of which is incorporated by reference.

### STATEMENT OF GOVERNMENT SUPPORT

The invention disclosed herein was made with U.S. Government support from National Institute of Diabetes and Digestive and Kidney Diseases Grants P01-DK060037, K01-DK67270, R21DK079594 and R01DK077665, National Institutes of Health, U.S. Department of Health and Human Services. Accordingly, the U.S. Government has certain rights in this invention.

### FIELD OF THE INVENTION

The present invention relates to assays for erectile and bladder dysfunction and vascular health (e.g., cardiovascular disease, hypertension), where the assays detect the expression of one or more of the human Vcsa1 gene family. The assays are also useful for monitoring the efficacy of treatment of these disorders.

### BACKGROUND OF THE INVENTION

Throughout this application various publications are referred to in parenthesis. Citations for these references may be found at the end of the specification immediately preceding the claims. The disclosures of these publications are hereby incorporated by reference in their entireties into the subject application to more fully describe the art to which the subject application pertains.

A National Institutes of Health consensus panel defined erectile dysfunction (ED) as the inability to achieve or maintain erection sufficient for satisfactory sexual performance. The development of ED is multifactorial and there are several risk factors for ED. Depending on the cause ED can be broadly classified as organic, psychogenic or mixed (Lizza and Rosen 1999). Because of the multifactorial nature of ED, it has been difficult to identify a universal molecular marker for organic ED. Two of the most common risk factors for organic ED are diabetes and aging (Korenman 2004). Diabetic men are three times as likely to have ED as nondiabetic men and men 50 to 90 years old are at 10 times greater risk for ED than those younger than 50 years (Shabsigh et al. 2005).

Penile erection is a neurovascular process that relies on a concerted action of the nervous system, the vascular system and cavernous smooth muscle tissues. Decreased tone (relaxation) of the penile vasculature and the smooth muscle tissue of the corpora cavernosa results in penile erection (tumescence) (Andersson 2003). Many of the steps of neurotransmission, impulse propagation, and intracellular transduction of neural signals that accompany this process are only partly understood. At the biochemical level, normal smooth muscle tone is achieved through a balance of biochemical pathways that together regulate contraction or relaxation of the smooth muscle via myosin-driven actin filament sliding. The central regulator of smooth muscle contraction is the state of myosin light chain (MLC) phosphorylation. Contractile stimuli lead to an increase in [Ca²⁺]ᵢ activating the Ca²⁺-calmodulin-dependent myosin light chain kinase (MLCK). This in turn phosphorylates the 20-kDa regulatory light chain (MLC) of SM myosin (SMM) at Ser¹⁹ and shows a direct correlation with an increase in actin-activated ATP hydrolysis and cross-bridge cycling needed for generation of force (Hai and Murphy 1988, Samuel et al. 1990). Activation by a relaxation stimulus causes the level of cytosolic calcium to decrease, thereby inactivating MLCK. The myosin is dephosphorylated by smooth muscle myosin phosphatase (SMMP) (Hartshorne et al. 1998), leading to smooth muscle relaxation.

ED is attributable to inability of the cavernous smooth muscle tissue to undergo relaxation. It might be expected that different types of ED that have overlapping pathophysiological mechanisms may also have common biochemical pathways contributing to ED. However, microarray studies of different models of ED, such as diabetes (Sullivan et al. 2005) and post-radical prostatectomy models (User et al. 2003), only serve to highlight that ED involves changes in a diverse set of molecular pathways that do not overlap. The Vcsa1 transcript (variable coding sequence a1 gene, also known as a submandibular rat 1 gene) is down-regulated in a neurogenic (bilaterally ligated cavernous nerve) ED model (User et al. 2003). However, corresponding changes in Vcsa1 were not reported in a rat model of diabetic ED (Sullivan et al. 2005). Thus, these types of studies have so far only served to highlight that ED involves changes in a diverse set of molecular pathways.

The Vcsa1 gene that encodes the rat SMR1 protein is a member of the variable coding sequence multigene family. This family is characterized by extensive sequence variation in the coding region of the genes, and family members are located proximally and share a common gene structure (Rosinski-Chupin et al. 1995). Most VCS genes are coded by three exons. The first exon contains 5′ UTR, the second exon contains 5′ UTR and the coding region for most of the signal peptide, and the third exon contains the remainder of the coding region and the 3′UTR (Rosinski-Chupin et al. 1990). The signal peptide and N- and C-termini are well conserved between family members, while the central region of the coding sequence is hypervariable (Tronik-LeRoux et al. 1994). There is a higher than average rate of non-synonymous mutations in the hypervariable region of these genes that confers significant variation in the amino acid content and structure of the resulting proteins (Courty et al. 1996). The introduction of a premature stop codon in the gene products of some family members results in variations in the length of the C-terminus, adding another level of diversity between proteins (Courty et al. 1996). Differences in protein sequence between family members modify the number and position of dibasic motifs that regulate the cleavage of the resulting prohormones, thus yielding different peptide products on cleavage (Courty et al. 1996).

Individual VCS genes are also regulated by alternative splicing and alternative utilization of polyadenylation sites (Courty et al. 1996). In the case of Vcsa1, four gene products containing the same coding sequence but with divergent 3′ UTRs are produced that may be subject to differential post-transcriptional regulation (Courty et al. 1995). In addition, a fifth transcript that results from the utilization of an additional exon produces a protein with an unrelated sequence and presumably unrelated function (Courty et al. 1995).

The VCS genes have most likely evolved from a common ancestor by tandem gene duplication (Courty et al. 1996). Insertions, deletions, and mutations have resulted in the presence of many related but functionally distinct genes. There are two subclasses of the VCS family, the VCSA subgroup of preprohormones to which the gene encoding SMR1 belongs, and the VCSB subgroup of proline-rich salivary proteins (Courty et al. 1994). In Rattus norvegicus, there are 3 members of the VCS family clustered on chromosome 14, in the p21-p22 region (Rosinski-Chupin et al. 1995). These include 2 members of the VCSA subgroup, Vcsa1 and Vcsa2, and one member of the VCSB subgroup, Vcs-beta1, whose protein product may be a secreted prohormone, but is likely functionally unrelated to the VCSA family members (Courty et al. 1994).

The VCS genes are found exclusively in mammals, and the gene cluster is relatively well conserved in all mammals studied (Rijnkels et al. 2003). In the mouse, a cluster on chromosome 5 contains Vcs2, a VCSA subgroup member, and Vcs1, a VCSB subgroup member (Sensorale-Pose et al. 1997, Tronik-LeRoux et al. 1994). All placental mammals studied have genes related to the proline-rich VCSB family, whereas the VCSA family to which the gene for SMR1 belongs appears to have emerged more recently exclusively in rodents (Rougeot et al. 1998). Human members of the VCSB family are located in a cluster on chromosome 4q13.3 and include PROL1, submaxillary gland androgen regulated protein, homolog B (SMR3B) (PROL3), and submaxillary gland androgen regulated protein, homolog A (SMR3A) (PROL5). These genes encode salivary and lacrimal secreted proline-rich proteins (Dickinson et al. 1996, Isemura 2000, Isemura and Saitoh 1997).

The VCS gene cluster is located within a larger cluster that contains the statherin/histatin salivary proteins, milk caseins, and enamel matrix proteins (Kawasaki and Weiss 2003, Rijnkels et al. 2003). The clustering of these gene families is conserved among mammals, and the genes in these families share similar structural features and are all generally expressed in glandular, secretory tissues (Kawasaki and Weiss 2003). Recent analysis has suggested that the members of all of these clustered gene families originated from a common ancestor, the gene for the secretory calcium-binding phosphoprotein SPARCL1 (Kawasaki et al. 2004, 2006).

The VCS gene family consists of a number of related but diverse genes that encode proteins with a variety of functions. These proteins are generally expressed in salivary glands of mammals, and most are secreted proteins that are subject to post-translational processing into biologically active small peptide fragments. The SMR1 protein product of the rat Vcsa1 gene is cleaved into at least two biologically active peptides, sialorphin (QHNPR) (SEQ ID NO:25) and SGPT (TDIFEGG) (SEQ ID NO:26). The N-terminal QHNPR sequence is conserved in all products of the rat VCSA family members and similar but not identical sequences are present in the mouse Vcs2 products (MSG1-2) (Rougeot et al. 1998). In contrast, the C-terminal TDIFEGG (SEQ ID NO:26) sequence is not present in any other product of the rat or mouse VCSA subfamily due to mutation or truncation of the C-terminus (Rougeot et al. 1998). Since the VCSA subgroup of genes is not present in non-rodent mammals, it might be predicted that proteins with these biologically active motifs would not be present in humans. However, both sialorphin and SGPT are biologically active in many species that do not possess these genes, including humans. Recently, it has been demonstrated that the human VCSB gene PROL1 encodes a protein that contains a QRFSR (SEQ ID NO:23) motif (opiorphin) that is functionally equivalent to rat sialorphin (Wisner et al. 2006).

Vcsa1 encodes a precursor protein that gives rise to three peptide products, including an undecapeptide, a hexapeptide and a pentapeptide (Rougeot et al. 1994). The final mature peptide is the pentapeptide, named sialorphin. Several roles for Vcsa1 have been proposed. There is evidence that sialorphin has a role in male rat sexuality since there is 100 to 500 times greater circulating sialorphin peptide levels in adult male rats than in females and dorsal tail injection of sialorphin modulates male rat sexual behavior (Messaoudi et al. 2004; Rosinski-Chupin et al. 2001). Other studies have shown that sialorphin is an inhibitor of rat membrane-bound neutral endopeptidase (NEP) (Rougeot et al. 2003) displaying analgesic activity, and binding studies have suggested a link between the circulating peptides and mineral transport (Rougeot et al. 1997). NEP plays an important role in nervous and peripheral tissues, as it turns off several peptide-signaling events at the cell surface. It has been demonstrated that sialorphin prevents spinal and renal NEP from breaking down two of its physiologically relevant substrates, substance P and Met-enkephalin in vitro (Rougeot et al. 2003). A similar peptide to sialorphin (called opiorphin) was recently identified in human saliva, and also acts as an NEP inhibitor (Wisner et al. 2006). The synthetic NEP inhibitors, phosphoramidon and thiorphan, have been shown to enhance C-type natriuretic peptide (CNP)-induced relaxation of porcine isolated coronary artery smooth muscle (Marton et al. 2005). Interestingly, CNP has also been suggested to play a role in erectile function (Walther and Stepan 2004). CNP binds to corporal smooth muscle guanylyl cyclase B receptor present in both rabbits and rats, and it was demonstrated that CNP could cause relaxation of isolated rabbit corporal smooth muscle tissue (Kim et al. 1998, Kuthe et al. 2003). There is considerable sexual dimorphism in the regulation of Vcsa1 gene expression, androgens causing expression of 100- to 500-fold higher mature peptide hormone levels in adult males compared with adult females (Rosinski-Chupin et al. 1993). Homologues with close identity to the Vcsa1 gene were reported in mice (mSG1, mSG2 and mSMR2), cows (bovine P-B) and humans (hSMR3A) (Isemura et al., 1994, 2004).

Type I diabetes is a metabolic disorder in which hyperglycemia and other metabolic defects lead to a significant morbidity and mortality through cardiovascular disease, stroke, blindess, nerve impairment and amputations. Better assessments of disease progression are needed for patient care and the development of therapeutics. This has resulted in the NIH NIDDK issuing the RFA DK-06-004 entitled “Biomarker Development for Diabetic Complications” for exploratory and developmental research on biomarkers for the microvascular and macrovascular complications of diabetes.

Erectile dysfunction is a common complication of diabetes, with diabetic men being three times as likely to develop ED as non-diabetic men (Korenman 2004, Shabsigh et al. 2005). ED is essentially a vascular disease, in which the blood vessels of the penis have heightened tone restricting the flow of blood into the penis that is required for an erection (Andersson 2001, 2003; Shabsigh and Anastasiadis 2003). Diabetes is also a risk factor for the development of cardiovascular disease (CVD), which is the major cause of death in type I and II diabetics (Marshall and Flyvbjerg 2006). It is increasingly being recognized that ED is an important marker of vascular disease, and there is growing evidence that both ED and CVD both share common mechanisms of development through vascular endothelial dysfunction (Eaton et al. 2006, Kirby et al. 2005, Muller and Mulhall 2006). Indeed, it has been recommended that patients with ED should be investigated for CVD (Burnett et al. 2006, Feldman et al. 1994, Grover et al. 2006, Kupelian et al. 2006, Stuckey et al. 2006, Thompson et al. 2005). It is estimated more than 600,000 men aged 40 to 69 years in the United States develop ED and with the availability of effective pharmacotherapy an increasing number of men approach their physicians for treatment. These men, who would not otherwise seek medical examination, represent a huge potential for prescreening and prevention of more serious vascular complications.

Thus, there is a need for the identification of biomarkers for both vascular complications, as indicated by the RFA DK-06-004 issued by the NIH-NIDDK for “Biomarker Development for Diabetic Complications,” and for dysfunctions such as erectile dysfunction and bladder dysfunction. Estimates of the incidence of ED range from 15 million to 30 million. According to the National Ambulatory Medical Care Survey (NAMCS), for every 1,000 men in the United States, 7.7 physician office visits were made for ED in 1985. By 1999, that rate had nearly tripled to 22.3. The increase happened primarily because of the introduction and associated publicity of the oral drug sildenafil citrate (Viagra®) in March 1998. NAMCS data on new drugs show an estimated 2.6 million mentions of Viagra® at physician office visits in 1999, and one-third of those mentions occurred during visits for a diagnosis other than ED. Diabetes has an incidence of approximately 1 in 340 or 0.29% in the USA (about 16 million people), and is increasing at a rate of about 798,000 people per year. Screening patients in just these two groups could therefore represent around 31 to 46 million people in the U.S.A. alone.

### SUMMARY OF THE INVENTION

The present invention provides methods for determining whether a subject has erectile dysfunction, bladder dysfunction, cardiovascular disease or hypertension, where the methods involve determining the expression of a human Vcsa1 family member in the subject, wherein decreased expression of the human Vcsa1 family member is indicative of erectile dysfunction, bladder dysfunction, cardiovascular disease or hypertension in the subject.

The invention also provides methods for monitoring the efficacy of treatment of a subject for erectile dysfunction, bladder dysfunction, cardiovascular disease or hypertension, where the methods involve determining the expression of a human Vcsa1 family member in a subject undergoing treatment for erectile dysfunction, bladder dysfunction, cardiovascular disease or hypertension, wherein increased expression of the human Vcsa1 family member is indicative of effective treatment or wherein decreased expression of the human Vcsa1 family member is indicative of a need to continue treatment.

### BRIEF DESCRIPTION OF THE FIGURES

FIG. 1. Expression of Vcsa1 transcripts normalized to GAPDH were analyzed using the comparative crossing threshold (Cₜ) method. Vcsa1 gene expression was determined in corpora, bladder, urethra and ureter in control (nondiabetic), DM (STZ-diabetic) 1-week, or DM 2-month animals. The 2-month control bladder tissue was used as the calibrator tissue (set as 1). N=number of animals used in experiments. Each quantitative PCR measurement was performed in duplicate for each tissue. The bars represent the mean comparative expression of the gene, and the error bars the standard deviation. *=Significantly different expression of Vcsa1 compared to the control (nondiabetic) value for a particular tissue (P<0.5).

FIG. 2. Expression of Vcsa1 transcripts normalized to GAPDH were analyzed using the comparative crossing threshold (Cₜ) method. Vcsa1 gene expression was determined in corpora, bladder, urethra, and ureter in young animals and retired breeders. N=number of animals. Quantitative PCR was performed in duplicate for each tissue; the bar represents the mean with the expression of Vcsa1 in the bladder tissue of young animals used as the calibrator tissue (set as 1). Error bars represent the standard deviation. *=Significantly different expression of Vcsa1 compared to the value for tissue from the young animal (P<0.5).

FIG. 3A-3B. Histological analysis of corporal tissue sections 1 week after intracorporal injection of 80 μg pVAX-Vcsa1 (B) compared with untreated control tissue (A). Magnification is ×4 in lower panels and ×10 in upper panels.

FIG. 4A-4B. A, Results of a typical experiment in which (upper panel) intracorporal pressure (ICP) and (lower panel) blood pressure (BP) was measured after cavernous nerve (CN) electrostimulation at 0.75 and 4 mA before treatment with sialorphin and after various times of treatment. B, Average of ICP/BP measurements for seven animals. There seems to be a time-dependent increase in the effect of sialorphin on erectile function, increasing with time. A significant effect (p<0.05, Student's t test) on erectile function is seen at 35 to 45 min and 55 to 65 min after intracorporal injection of sialorphin for the lower level of stimulation (0.75 mA) and after 1 h for the higher level of stimulation (4 mA).

FIG. 5A-5B. A, Myograph of corporal strips contracted with 1 μM phenylephrine. Relaxation was induced by addition of C-type natriuretic peptide (CNP; 1 μM). Addition of sialorphin (1 μg/ml) further increased the rate of relaxation. The change in the rate of relaxation after the addition of 1 pg/ml sialorphin was derived from the slope of the recording as the change in tension (g) over time (minutes) (as shown in the figure). DMSO=dimethyl sulfoxide.

FIG. 6. The proposed pathway by which sialorphin causes smooth muscle relaxation and thereby improved erectile function. C-type natriuretic peptide (CNP) binds to its membrane receptor on corporal smooth muscle cells and activates guanylyl cyclase (GC-B). The CNP is degraded by neutral endopeptidase (NEP). However, in the presence of sialorphin (acting as an NEP inhibitor), the CNP has a prolonged effect, activating downstream mechanisms that result in smooth muscle relaxation, mediated by the secondary messenger cyclic guanine monophosphate (cGMP). Among these downstream activators are Maxi-K channels. Efflux of potassium from the cells causes hyperpolarization of the smooth muscle cell membrane, inhibiting influx of Ca²⁺ through calcium channels. Lowered intracellular calcium causes inactivation myosin light chain kinase (MLCK), thereby promoting smooth muscle relaxation. GTP=guanosine triphosphate; PKA=protein kinase A; PKC=protein kinase C; PKG=protein kinase G; MLC20=myosin light chain.

FIG. 7A-7B. Nucleotide sequence comparison of Vcsa1 and hSMR3A with consensus sequence (A) and amino acid sequence comparison of Vcsa1 and hSMR3A with consensus sequence (B). Underlined text indicates primers used to specifically amplify hSMR3A gene. SEQ ID NO: 18—Vcsa1 nucleotide sequence; SEQ ID NO: 22—Vcsa1 amino acid sequence; SEQ ID NO: 15—hSMR3A nucleotide sequence; SEQ ID NO: 19—hSMR3A amino acid sequence.

FIG. 8. Histological comparison of distal sections of normal penis and that of rat treated with 100 μg pVAX-hSMR3A. Reduced from ×4.

FIG. 9. hSMR3A transcript expression was analyzed using comparative crossing threshold (Ct) method, also known as 2^{-δδCt} method, which was applicable because SMR3A primer efficiency for generating products was close to that of housekeeping gene glyceraldehyde-3-phosphate dehydrogenase (GAPDH) used to normalize samples. Expression level is compared to that in patient 0A.

FIG. 10. Prol1 is down-regulated in human erectile dysfunction. Expression of Prol1 transcripts was analyzed using the comparative crossing threshold (Ct) method. The house keeping gene GAPDH was used to normalize samples. The patients are the same as described in Table 5. Level of expression is compared to Patient 0A.

FIG. 11A-11B. A: The time of the longest visually observed erection (in seconds) after initiation of the experiment prior to measurement of ICP/BP. B: The ICP/BP response after CN electrostimulation at 0.75, 4 and 10 mA after various treatments to improve erectile function. 1000 μg pVAX-hSlo (hSlo) was intracorporally injected one month prior to studies where indicated (+) and 2.5 mg/kg Cialis® was administered orally 2 h prior to the experiment as indicated (+). *=significantly different from untreated control (P<0.5)**=combined treatment (cialis+hSlo) is significantly different from hSlo alone (P<0.5)

FIG. 12. The results for the RT-PCR analysis of the Vcsa1 and Slo gene (MaxiK) transcripts after treatment are with pVAX-hSlo and Cialis® (as described in FIG. 11A-11B). At least 4 animals in each group were analyzed in duplicate and the results for all 8 quantitative PCRs averaged. Each quantitative PCR measurement was performed in duplicate for each tissue. The corporal tissue from untreated animals was used as calibrator tissue. *=Significantly different expression of Vcsa1 compared to the control (nondiabetic) value (P<0.5). **=Significantly different expression of Vcsa1 compared to the animals receiving one treatment (P<0.5).

### DETAILED DESCRIPTION OF THE INVENTION

The present invention provides a method for determining whether a subject has erectile dysfunction, bladder dysfunction, cardiovascular disease or hypertension, the method comprising determining the expression of a human Vcsa1 family member in the subject, wherein decreased expression of the human Vcsa1 family member is indicative of erectile dysfunction, bladder dysfunction, cardiovascular disease or hypertension in the subject. Preferably, the amount of decreased expression of the human Vcsa1 family member is indicative of the degree of pathology of erectile dysfunction, bladder dysfunction, cardiovascular disease or hypertension.

The invention also provides a method for monitoring the efficacy of treatment of a subject for erectile dysfunction, bladder dysfunction, cardiovascular disease or hypertension, the method comprising determining the expression of a human Vcsa1 family member in a subject undergoing treatment for erectile dysfunction, bladder dysfunction, cardiovascular disease or hypertension, wherein increased expression of the human Vcsa1 family member is indicative of effective treatment or wherein decreased expression of the human Vcsa1 family member is indicative of a need to continue treatment. Preferably, the amount of decreased expression of the human Vcsa1 family member is indicative of the degree of pathology of erectile dysfunction, bladder dysfunction, cardiovascular disease or hypertension. Preferably, the amount of increased expression of the human Vcsa1 family member is indicative of the degree of the effectiveness of treatment of erectile dysfunction, bladder dysfunction, cardiovascular disease or hypertension.

Decreased expression of the human Vcsa1 family member can be measured relative to the expression of the human Vcsa1 family member in control subjects without erectile dysfunction, bladder dysfunction, cardiovascular disease or hypertension.

Erectile dysfunction can be classified as organic, psychogenic or mixed. As used herein, “organic” erectile dysfunction means erectile dysfunction having a physiological, non-psychological cause. The erectile dysfunction can be associated with diabetes and/or aging.

The assays described herein can be used to delineate between erectile dysfunction caused by psychogenic problems, where levels of the human Vcsa1 family member are normal, and erectile dysfunction caused by organic (physiological) problems, where levels of the human Vcsa1 family member are decreased.

The human Vcsa1 family member can be obtained from a sample from the subject, such as a blood, saliva or corpus cavernosum tissue sample.

Preferably, the human Vcsa1 family member is hSMR3A, hSMR3B or PROL1. Preferably, SMR3A (human) has the nucleotide sequence set forth in SEQ ID NO:15:

atgaaatcactgacttggatcttgggcctttgggctcttgcagcgtgtttcacacctggtgagagtcaaa
gaggccccaggggaccatatccacctggaccactggctcctcctcctccaccatgttttccttttggaac
aggatttgttccaccaccccatcctccaccctatggtccagggagatttccaccacccctttctccaccc
tatggtccagggagaatcccaccatcccctcctccaccctatggtccagggagaattcaatcacactctc
ttcctcctccttatggcccaggttatccacagccaccttcccaaccaagaccctatccacctggacctcc
atttttccctgtaaattctccaactgatcctgccctccctactcctgcaccctaa.

Preferably, SMR3B (human) has the nucleotide sequence set forth in SEQ ID NO:16:

atgaaatcactgacttggatcttgggcctttgggctcttgcagcgtgtttcacacctggtgagagtcaaa
gaggccccaggggaccatatccacctggaccgctggctcctcctcaaccttttggcccaggatttgttcc
accacctcctcctccaccctatggtccagggagaatcccacctcctcctcccgcaccctatggtccaggg
atatttccaccaccccctcctcaaccctaa.

Preferably, PROL1 (human) has the nucleotide sequence set forth in SEQ ID NO:17:

atgaaattaactttcttcttgggcctgttggctcttatttcatgtttcacacccagtgagagtcaaagat
tctccagaagaccatatctacctggccagctgccaccacctccactctacaggccaagatgggttccacc
aagtcccccacctccctatgactcaagacttaattcaccactttctcttccctttgtcccagggcgagtt
ccaccatcttctttctctcgatttagccaagcagtcattctatctcaactctttccattggaatctatta
gacaacctcgactctttccgggttatccaaacctacatttcccactaagaccttactatgtaggacctat
taggatattaaaacccccatttcctcctattcctttttttcttgctatttaccttcctatctctaaccct
gagccccaaataaacatcaccaccgcagatacaacaatcaccacaaatccccccaccactgcaacagcaa
ccaccagcacttccacaaaacccacaatgacgatcagctcctcaacagtacctatctcttcaacaccaga
gcctgccacctccatatcagcagcaacccccgcagcatctactgaaaatactactcaaattctcgccaac
cgtcctcacacagtattgctcaatgccactgtccaagttacgacttccaaccaaactatattaagcagcc
cagcctttaaaagtttttggcaaaaactctttgccatttttggtta.

Expression of the human Vcsa1 family member can be determined by measuring mRNA expression and/or by measuring protein expression and/or expression of a peptide portion of a human Vcsa1 family member protein.

Preferably, SMR3A protein (human) has the amino acid sequence set forth in SEQ ID NO:19:

| | |
|---|---|
| 1 | mksltwilgl walaacftpg esqrgprgpy ppgplapppp prfpfgtgfv ppphpppygp |
| 61 | grfppplspp ygpgrippsp pppygpgriq shslpppygp gypqppsqpr pyppgppffp |
| 121 | vnsptdpalp tlap. |

Preferably, SMR3B protein (human) has the amino acid sequence set forth in SEQ ID NO:20:

| | |
|---|---|
| 1 | mksltwilgl walaacftpg esqrgprgpy ppgplappqp fgpgfvpppp pppygpgrip |
| 61 | ppysctpnmn ncsrchhhhk rhhypcnycf cypkyefqhc fqetft. |

Preferably, PROL1 protein (human) has the amino acid sequence set forth in SEQ ID NO:21:

| | |
|---|---|
| 1 | mkltfflgll aliscftpse sqrfsrrpyl pgqlpppply rprwvppspp ppydsrlnsp |
| 61 | lslpfvpgrv ppssfsrfsq avilsqlfpl esirqprlfp gypnlhfplr pyyvgpiril |
| 121 | kppfppipff laiylpisnp epqinittad ttittnpptt atattststk ptmtissstv |
| 181 | pisstpepat sisaatpaas tenttqilan rphtvllnat vqvttsnqti lsspafksfw |
| 241 | qklfaifg. |

An example of a human Vcsa1 family member peptide is opiorphin (QRFSR) (SEQ ID NO:23), which is derived from Prol1.

The nucleotide sequence for rat Vcsa1 is (SEQ ID NO:18):

| | |
|---|---|
| atgaagtcac tgtatttgat ctttggcctg tggatccttc tagcatgctt ccagtcaggt | 60 |
| gagggtgtca gaggcccaag aagacaacat aatcctagaa gacaacaaga tccttcaact | 120 |
| cttcctcatt atcttggtct tcagcctgat cccaatggtg gacaaatagg agtaacaatc | 180 |
| actataccct taaatcttca accacctcgt gttcttgtta atcttcccgg ttttatcact | 240 |
| ggaccaccat tggttgtaca aggtaccact gaatatcaat atcagtggca gctaactgct | 300 |
| ccagacccta cacctctaag caatcctcct actcaacttc tttccacaga acaagcaaat | 360 |
| acaaaaacag atgccaaaat ctccaacact actgcgacta cccaaaattc cactgatatt | 420 |
| tttgaaggtg gtggcaaata a | 441. |

The rat Vcsa1 gene product has the sequence (SEQ ID NO:22):

| | |
|---|---|
| MKSLYLTFGL WTLLACFQSG EGVRGPRRQH NPRRQQDPST LPHYLGLQPD PNGGQIGVTI | 60 |
| TKTDAKISNT TATTQNSTDI FEGGGK | 86. |

The expression of the human Vcsa1 family member may be detected in vitro or in vivo. Where expression is detected in vitro, a sample of blood, saliva, tissue or cells from the subject may be removed using standard procedures, including biopsy and aspiration. Cells which are removed from the subject may be analyzed using immunocytofluorometry (FACS analysis). The expression of the human Vcsa1 family member may be detected by detection methods readily determined from the known art, including, without limitation, immunological techniques such as Western blotting, hybridization analysis, fluorescence imaging techniques, and immunoassay such as a radioimmunoassay (RIA) or an enzyme linked immune assay (ELISA).

The blood, saliva, tissue or cell sample can be assayed using an agent that specifically binds to the human Vcsa1 family member, such as, for example, an antibody, a peptide or an aptamer. As used herein, the term “antibody” encompasses whole antibodies and fragments of whole antibodies wherein the fragments specifically bind to Vcsa1. Antibody fragments include, but are not limited to, F(ab′)₂ and Fab′ fragments and single chain antibodies. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. Antibodies may be produced by techniques well known to those skilled in the art. Polyclonal antibody, for example, may be produced by immunizing a mouse, rabbit, or rat with purified polypeptides encoded by the human Vcsa1 family member. Monoclonal antibody may then be produced by removing the spleen from the immunized mouse, and fusing the spleen cells with myeloma cells to form a hybridoma which, when grown in culture, will produce a monoclonal antibody. The antibody can be, e.g., any of an IgA, IgD, IgE, IgG, or IgM antibody. The IgA antibody can be, e.g., an IgA1 or an IgA2 antibody. The IgG antibody can be, e.g., an IgG1, IgG2, IgG2a, IgG2b, IgG3 or IgG4 antibody. A combination of any of these antibodies subtypes can also be used. The antibody can be a human antibody or a non-human antibody such as a goat antibody or a mouse antibody or a rabbit antibody. Antibodies can be “humanized” using standard recombinant DNA techniques.

Aptamers are single stranded oligonucleotides or oligonucleotide analogs that bind to a particular target molecule, such as a protein. Thus, aptamers are the oligonucleotide analogy to antibodies. However, aptamers are smaller than antibodies. Their binding is highly dependent on the secondary structure formed by the aptamer oligonucleotide. Both RNA and single stranded DNA (or analog) aptamers can be used. Aptamers that bind to virtually any particular target can be selected using an iterative process called Systematic Evolution of Ligands by EXponential enrichment (SELEX).

The agent that specifically binds to the human Vcsa1 family member may be labeled with a detectable marker. Labeling may be accomplished using one of a variety of labeling techniques, including peroxidase, chemiluminescent, and/or radioactive labels known in the art. The detectable marker may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as, for example, ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

The expression of the human Vcsa1 family member may be detected through hybridization analysis of nucleic acid extracted from a blood, saliva, tissue or cell sample from the subject using one or more nucleic acid probes which specifically hybridize to nucleic acid encoding the human Vcsa1 family member. The nucleic acid probes may be DNA or RNA, and may vary in length from about 8 nucleotides to the entire length of the human Vcsa1 family member. Hybridization techniques are well known in the art, see e.g. Sambrook and Russell (2001). The probes may be prepared by a variety of techniques known to those skilled in the art, including, without limitation, restriction enzyme digestion of the human Vcsa1 family member nucleic acid; and automated synthesis of oligonucleotides with a sequence that corresponds to selected portions of the nucleotide sequence of the human Vcsa1 family member, using commercially-available oligonucleotide synthesizers, such as the Applied Biosystems Model 392 DNA/RNA synthesizer. Combinations of two or more nucleic acid probes, corresponding to different or overlapping regions of the human Vcsa1 family member, may be used to assay a diagnostic sample for expression of the human Vcsa1 family member.

The nucleic acid probes may be labeled with one or more detectable markers. Labeling of the nucleic acid probes may be accomplished using a number of methods known in the art (e.g., nick translation, end labeling, fill-in end labeling, polynucleotide kinase exchange reaction, random priming, or SP6 polymerase) with a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)).

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example I

### Vcsa1 (SMR-1) as a Marker for Erectile Dysfunction

### Materials and Methods

Diabetic animal model. The animal model for diabetes used in these experiments is STZ-induced diabetes in male rats (Christ et al 2004). Diabetes was induced in F-344 rats (Taconic Farms, Germantown, N.Y.; 8-10 weeks old and weighing 200-240 g) via a single intraperitoneal injection of streptozotocin (STZ) (35 mg/kg) dissolved in citrate buffer (0.6 M citric acid/0.08 M Na₂HPO₄; pH 4.6). Control (nondiabetic) animals received an injection of vehicle only. Streptozotocin-diabetic rats had blood glucose levels of 250 mg/dl or more and urine glucose levels 1000 mg/dl or more. One week or 2 months after onset of diabetes, animals were analyzed for intracorporal pressure/blood pressure (ICP/BP) response and then killed by placement within a CO₂ gas chamber; tissues of interest (corpora, bladder, urethra and ureter) were immediately flash frozen in liquid nitrogen and were stored at −70° until RNA preparation. The numbers of animals used in these experiments are shown in Table 1.

Aging animal model. Experiments were carried out on 4- to 5-month-old (young) male rats (approximately 275 g) and 9- to 10-month-old (old) retired breeder male Sprague-Dawley rats weighing more than 500 g (as described in Melman et al. 2003). Animals from the two age groups were analyzed for ICP/BP response and then were killed by placement within a CO₂ gas chamber; tissues of interest (corpora, bladder, urethra and ureter) were flash frozen in liquid nitrogen and were stored at −70° until RNA preparation. Numbers of animals used in these experiments are shown in Table 2.

Neurogenic model. The neurogenic ED model are rats with bilaterally ligated cavernous nerves (CNs). Four adult 120-day-old male Sprague-Dawley rats underwent open surgical bilateral CN ligation as previously described (User et al. 2003). CNs were identified bilaterally on the lateral aspect of the prostate. A portion of the nerve on each side then was sharply dissected free of the surrounding tissues. Direct electrostimulation of CN on each side was carried out at 6 mA using bipolar hook electrodes. Quality of erection was noted visually on a three point scale following stimulation of CN on each side of the prostate (NE=no erection, PE=partial erection, FE=full erection). Then, the CN on each side was ligated at the most proximal point of the isolated CN portion using 3-0 silk tie. The operative site then was closed, and the rats were allowed to recover from surgery. After the period of 9 days, the second open surgical procedure was performed. Again, CNs were identified bilaterally and the ligated portion of each CN was dissected free of the surrounding tissues. The dissection was carried approximately 1 cm proximal to the point of ligation. Electrostimulation of each CN was performed proximal to the point of ligation using the same stimulation as used during the first operation. No erectile response was visualized in any of the rats.

All study protocols were approved by the animal use committee and internal review board at the Albert Einstein College of Medicine.

Cavernosometry: Determination of intracavernosal pressure response to stimulation of the cavernous nerve. Animals were anesthetized by intraperitoneal injection of pentobarbital sodium (35 mg/kg). An incision was made in the perineum, and a window was made in the ischiocavernosus muscle to expose the corpus cavernosum. The cavernous nerves were identified adjacent to the prostate gland. Direct electrostimulation of the cavernous nerve was performed with a delicate stainless steel bipolar hook electrode attached to the multijointed clamp. Each probe was 0.2 mm in diameter; the two poles were separated by 1 mm. Monophasic rectangular pulses were delivered by a signal generator (custom-made and with built-in constant current amplifier). Stimulation parameters were as follows: frequency, 20 Hz; pulse width, 0.22 ms; duration, 1 min. Current was applied at 0.75 and 4 mA. The changes in ICP and systemic BP were recorded at each level of neurostimulation. The mean ICP/BP, standard deviation, and analysis of variance were calculated for each of the treatment groups.

Gene transfer experiments. Microinjection of vectors/plasmids into rat corporal tissue was performed essentially as previously described (Christ et al. 1998, 2004; Melman et al. 2003). Animals were anesthetized by an intraperitoneal injection of pentobarbital sodium (35 mg/kg). An incision was made through the perineum, the corpus spongiosum was identified, and a window was made in the corpus spongiosum for identification of the corpus cavernosum. All microinjections consisted of a single bolus injection of naked plasmid DNA into the corporal tissue, made using an insulin syringe. The final volume of all microinjections was 150 μl. The plasmid pVAX is commercially available (Invitrogen, Carlsbad, Calif.). Construction of the plasmid pVAX-hSlo has been described previously (Melman et al. 2003). pVAX-Vcsa1 was generated by PCR amplification of total cDNA from rat corpora using the forward primer 5′-CAAGGGGCTACCAAAGATGAAG-3′ (SEQ ID NO:1) and the reverse primer 5′-CCAAAAGGAATTTATTATTTGC-3′ (SEQ ID NO:2). Products of the reaction were separated on an agarose gel and DNA extracted from a band of the expected size and sub-cloned into pVAX. The PCR product was sequenced to confirm accurate amplification of the Vcsa1 gene.

Quantitative PCR. Total RNA was extracted from frozen tissue with TRIzol according to the manufacturer's instruction. Briefly, approximately 50 mg tissue was added to 1 ml TRIzol reagent and homogenized using a polytron homogenizer (Brinkman, Westbury, N.Y.) for 30 s. The homogenized tissues were incubated for 5 min at room temperature followed by addition of 200 μl of chloroform. After mixing, the aqueous phases were separated by centrifugation (12000×g for 15 min) at 4° C. and then were transferred to a clean tube. The RNA was precipitated from the aqueous phase by addition of isopropyl alcohol and pelleted by centrifugation at 12000×g for 15 min at 4° C., washed once with 75% ethanol, and again pelleted at 12000×g for 15 min. The ethanol was aspirated and the RNA pellet was dissolved in sterile water. One microgram total RNA was reverse-transcribed to first-strand cDNA primed with Oligo(dT) using the Superscript (Invitrogen) First-Strand Synthesis System for real-time PCR. RNA was denatured for 5 min at 65° C. and immediately cooled on ice. Then RNA was combined with the Superscript II RT, 40 units of RNaseOUT recombinant ribonuclease inhibitor, and RT reaction buffer. cDNA synthesis was performed for 50 min at 42° C. RT products then were amplified using Sybr Green 2×PCR Master Mix (PE Applied Biosystems, Warrington, UK). Real-time quantitative PCR analysis was performed using the 7300 real-time PCR system (Applied Biosystems, Foster City, Calif.). The primers for Vcsa1 were: forward primer, 5′-GAGGGTGTCAGAGGCCC-3′ (SEQ ID NO:3); reverse primer, 5′-GAGCAGTTAGCTGCCACTGATA-3′ (SEQ ID NO:4) (nucleotides 147-163 and 364-384, respectively). Glyceraldehyde-3-phosphate Dehydrogenase (GAPDH; forward primer, 5′-GCCGCCTGCTTCACCACCTTCT-3′ (SEQ ID NO:5); reverse primer, 5′-GCATGGCCTTCCGTGTTCCTACC-3′) (SEQ ID NO:6) was used as an endogenous control. The PCR reactions for all samples were performed in 96-well plates, with 2 μl cDNA, 100 nM each primer, and 12.5 μl of SYBR Green in a 25-μl reaction volume. The cycling conditions were as follows: activation of SYBR Green DNA polymerase at 95° C. for 10 min, 40 cycles of denaturation at 95° C. for 15 s, annealing/extension at 60° C. for 1 min. Results from real-time PCR were presented as threshold cycles normalized to that of the GAPDH gene according to the method previously described (Livak and Schmittgen 2001). The relative quantified value for each target gene in diabetic rats compared with control rats is expressed as 2^{-(Ct-Cc)} (Ct and Cc are the mean threshold cycle differences after normalizing to GAPDH).

### Results

Erectile Capacity in Rats with 1 week and 2 Month STZ-Diabetes. Cavernosometry measurements were conducted to evaluate erectile capacity in rats after 1 week or 2 months of STZ-diabetes and were compared with responses obtained in nondiabetic rats. The mean amplitude of the ICP response was examined at 0.75 and 4 mA of current stimulation and was expressed as mean ICP/BP during 60 s of cavernous nerve stimulation. As shown in Table 1, after 1 week of STZ-diabetes there was no significant difference to non-diabetic animals in the basal ICP/BP response or following 0.75 and 4 mA electrostimulation. The ICP/BP values were in the range associated with normal erectile capacity. However, after 2 months of STZ-diabetes, although there was no significant difference in the basal level ICP/BP measurement, there was a significant decrease in the erectile function between the nondiabetic and STZ-diabetic animals at 0.75- and 4-mA levels of stimulation. The decrease in the ICP/BP measurements is consistent with the development of ED in the 2 month STZ-diabetic rat, as previously reported (Christ et al. 2004).

TABLE 1
Measurement of Erectile Function by ICP/BP in Nondiabetic,
1 week STZ-Diabetic and 2 month STZ-Diabetic Rats.

| | | Basal | | 0.75 mA | | 4 mA ICP/BP | |
|---|---|---|---|---|---|---|---|
| | | ICP/BP | | CP/BP | | Std | |
| Animal | N | Mean | Std Dev | Mean | Std Dev | Mean | Dev |
| Non-Diabetic | 4 | 0.063 | 0.025 | 0.577 | 0.116 | 0.623 | 0.166 |
| 1 Week Diabetic | 4 | 0.106 | 0.023 | 0.547 | 0.0324 | 0.597 | 0.15 |
| 2 month Diabetic | 5 | 0.023 | 0.023 | 0.34* | 0.195 | 0.464* | 0.119 |

ICP/BP ratio of animals after 1 week and 2 months of diabetes.
The cavernosal nerve was stimulated with 0.75 or 4 mA of current.
N = number of animals used in each group to determine the mean ICP/BP,
Std Dev = standard deviation.
*Significantly different from nondiabetic (P < 0.5).
One-way analysis of variance demonstrated that the ICP/BP of 2-month STZ-diabetic animals were statistically different from the nondiabetic and 1-week animals.

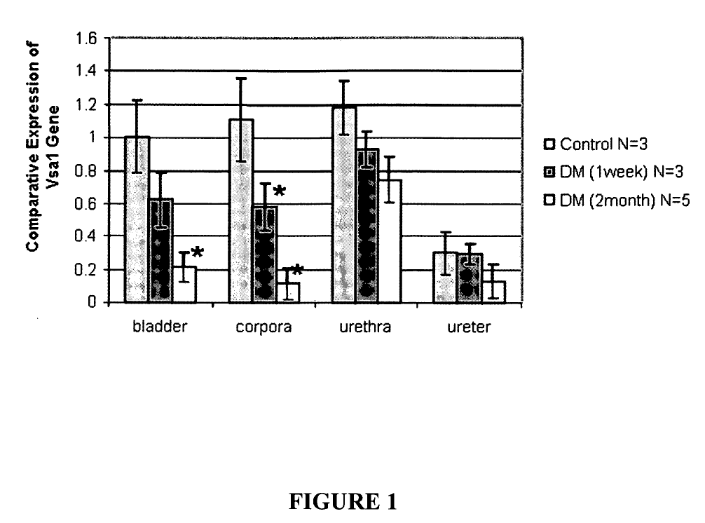

Real-time PCR for Vcs1a in diabetic nondiabetic animals. Using the ICP/BP data as criteria, experiments were designed to correlate the changes in expression of Vcsa1 with the onset of diabetes and development of ED. The 1-week time point represents a stage where animals have diabetes but no significant erectile impairment, whereas after 2 months of diabetes, there are obvious alterations in erectile capacity. Quantitative PCR was used to measure Vcsa1 expression in nondiabetic and 1-week and 2-month diabetic rats. Expression of transcripts was analyzed using the comparative crossing threshold (Cₜ) method (also known as the 2^{-(16)Ct} method (Livak and Schmittgen 2001)). This method was applicable because the efficiency of the primers in generating Vcsa1 product was found to be close to that of the housekeeping gene glyceraldehyde-3-phosphate dehydrogenase (GAPDH), which was used to normalize samples. In addition to corporal tissue, in order to gain insight into the changes that occur in Vcsa1 gene expression in other urogenital smooth muscle tissues, Vcsa1 gene expression was also analyzed in the bladder, urethra and ureter. The Cₜ values of the samples of interest were compared with bladder from control animals (nondiabetic), which were used as the calibrator tissue. Quantitative PCR demonstrated that after 1 week of diabetes, there was no significant change in the expression of Vcsa1 compared with nondiabetic animals in bladder, urethra and ureter (FIG. 1). In the corpora, there was a 50% decrease in expression of Vcsa1. However, after 2 months, there was an even greater decrease in Vcsa1 expression in the corpora, where there was a 10-fold decrease in Vcsa1 expression, and a significant decrease in the bladder of approximately 5-fold. These results show a correlation between decreased expression of Vcsa1 in the corpora and ICP/BP measurements.

Vcsa1 expression in an aging and neurogenic model of ED. A rodent model of age-related ED was also used to further evaluate the relationship between alterations in the ICP/BP values and expression of Vcsa1 levels in young rats as well as retired breeders. Experiments were carried out on 4- to 5-month-old (young) rats (approximately 275 g) and 9- to 10-month-old (old) retired breeder male Sprague-Dawley rats weighing more than 500 g. The ICP/BP response was examined at two levels of current stimulation, 0.75 and 4 mA. Although the basal ICP/BP measurements were similar in both young and retired breeders, as indicated in Table 2, the response to neurostimulation of the cavernous nerve was significantly diminished in old compared with young rats.

TABLE 2
Measurement of Erectile Function by ICP/BP
in Young and Retired Breeder Rats

| | | Basal | | | | 4 mA | |
|---|---|---|---|---|---|---|---|
| | | ICP/BP | | | | ICP/BP | |
| | | | Std | 0.75 mA ICP/BP | | | Std |
| Animal | N | Mean | Dev | Mean | Std Dev | Mean | Dev |
| Young | 7 | 0.1 | 0.057 | 0.597 | 0.143 | 0.631 | 0.101 |
| Retired | 6 | 0.086* | 0.057 | 0.213* | 0.179 | 0.245* | 01628 |
| Breeders | | | | | | | |

ICP/BP measurements from young and retired breeders.
N = number of animals used in each group to determine the mean ICP/BP,
Std Dev = standard deviation.
*Significantly different from young animal (P < 0.5).
One-way analysis of variance demonstrated that the ICP/BP of the young animals was statistically different from the retired breeders.

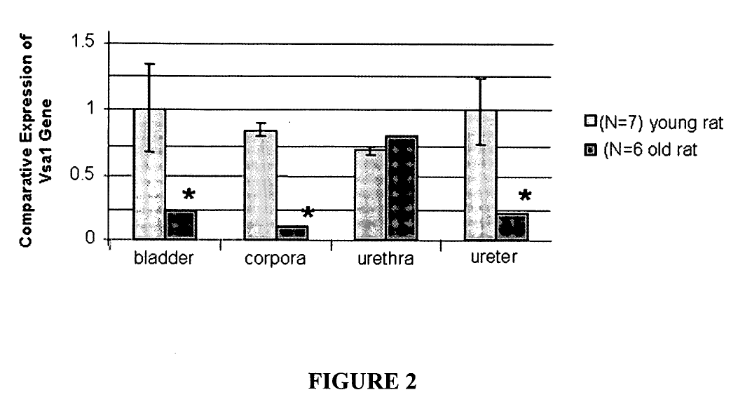

Immediately after cavernosometry measurements, rats were killed and RNA was prepared from corpora, bladder, urethra and ureter as described above. The relative expression of Vcsa1 determined in various urogenital tissues was compared between young and old animals (FIG. 2). In the older animals (which had decreased ICP/BP values and ED), there again was a significant decrease in Vcsa1 in the corpora of approximately 11-fold. Interestingly, its expression also was reduced in the bladder and ureter of the older animals by approximately 5-fold. There was little effect of aging on the expression of Vcsa1 in the urethra.

The cavernous nerves of rats were also bilaterally ligated (n=4) and, 9 days after surgery, real-time PCR was performed to analyze for the expression of Vcsa1 in corporal tissue compared with controls. Before surgery, all four animals in the study had visible erections when stimulated with 6 mA. However, 9 days after surgery, animals had no visible erection when stimulated with 6 mA. Real-time PCR experiments demonstrated that there was a 10-fold decrease (±0.8) in Vcsa1 expression in the corpora of CN-ligated animals compared with control animals (n=4).

The physiological role of Vcsa1 in erectile function. Overall, the above experiments demonstrate that in three rat models of ED (diabetic, age-related, and neurogenic) the Vcsa1 gene is downregulated. Therefore, this gene is a potential biomarker for ED. As such, it may play a direct role in modulating erectile capacity. To test this hypothesis, gene transfer experiments were performed using intracorporal injection in retired breeder rats of a plasmid (pVAX-Vcsa1) where expression is driven from the CMV (cytomegalovirus) promoter. As positive controls, another cohort of retired breeder animals were injected with pVAX-hSlo (Christ et al. 1998, Melman et al. 2003), which has been shown to ameliorate the age-related decline of erectile capacity observed in this rodent model. Finally, negative controls were run in parallel, where animals were injected with the backbone plasmid (pVAX). One week after injection, cavernosometry was performed and ICP/BP measurements were compared before corporal tissue excision and light microscopic examination. Results are shown in Table 3.

TABLE 3
Measurement of Erectile Function by ICP/BP in Retired Breeder Rats Following
Intracorporal injection of pVAX, pVAX-hSlo or pVAX-Vcsa1

| | | Basal | | 0.75 mA | | 4 mA | |
|---|---|---|---|---|---|---|---|
| | | ICP/BP | | ICP/BP | | ICP/BP | |
| Treatment | N | Mean | Std Dev | Mean | Std Dev | Mean | Std Dev |
| 100 μg pVAX | 8 | 0.0625 | 0.0198 | 0.218 | 0.118 | 0.334 | 0.178 |
| 100 μg pVAX-hSlo | 6 | 0.085 | 0.0288 | 0.687* | 0.0327 | 0.745* | 0.0632 |
| 80 μg pVAX-Vcsa1 | 4 | 0.148* | 0.0714 | 0.352* | 0.212 | 0.413* | 0.176 |
| 25 μg pVAX-Vcsa1 | 3 | 0.0967 | 0.0289 | 0.540* | 0.208 | 0.623* | 0.129 |
| 5 μg pVAX-Vcsa1 | 3 | 0.0733 | 0.0231 | 0.427* | 0.106 | 0.640* | 0.0458 |

Measurement of erectile function by ICP/BP in retired breeder rats after intracorporal injection of pVAX, pVAX-hSlo, or pVAX-Vcsa1.
The rats were 9 to 10 months old and weigh more than 500 g.
N = number of animals used in each group to determine the mean ICP/BP.
*one-way analysis of variance demonstrated that the ICP/BP of treated animals were statistically different from the control animals (pVAX) (P < 0.5).

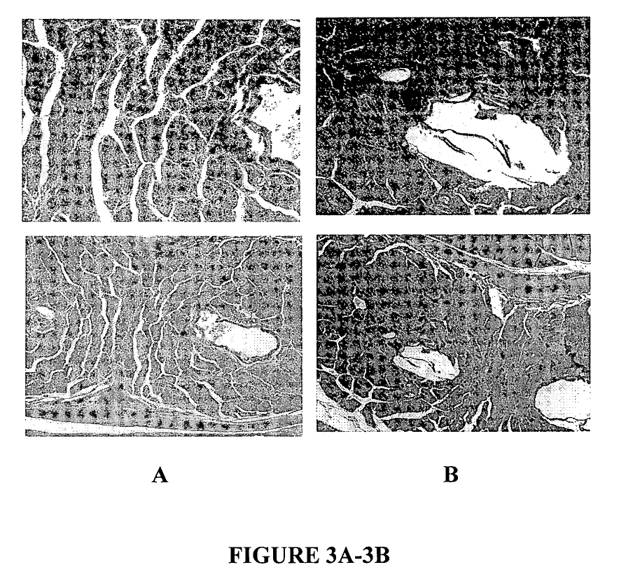

In animals treated with 80 μg pVAX-Vcsa1, there was a significant increase in the basal ICP/BP measurements compared with all the other control and experimental groups. After stimulation, there was a slight improvement in ICP/BP compared with the negative control, which did not result in a visible erection, except in one of the four animals that, after stimulation, exhibited visually priapism. Visual postmortem examination of the animals that were intracorporally injected with pVAX-Vcsa1 presented evidence of vascular congestion of the corporal tissue, suggesting that animals had experienced priapism. This was confirmed through microscopic examination of animals injected with 80 μg pVAX-Vcsa1 (FIG. 3). Trabecular interstitial edema, blood clot formation within the cavernae, and destruction of the endothelial lining could be seen with microscopy, suggesting that priapism had been severe in the 1 week between injection of the plasmid and termination of the experiment. Neither negative control animals nor animals injected with the same dose of pVAX-hSlo showed any signs of priapism.

At lower doses of pVAX-Vcsa1 (5 and 25 μg), there was a significant improvement in erectile response as indicated by the ICP/BP measurement compared with control animals (treated with the empty vector pVAX; Table 3). At the highest level of stimulation given in these experiments (4 mA), there was no significant difference in the ICP/BP value obtained after gene transfer of pVAX-Vcsa1 or pVAX-hSlo, which has been previously reported to restore erectile function in retired breeders and diabetic animals (Christ et al. 1998, 2004; Melman et al. 2003), and is undergoing clinical phase I trials for treatment of ED (Melman et al. 2005). However, after stimulation of the cavernous nerve, the time to return to basal ICP/BP in the animals treated with 5 and 25 μg pVAX-Vcsa1 was approximately twice as long as pVAX-hSlo. These results suggest that intracorporal injection of pVAX-Vcsa1 results in a disturbance in the normal efflux of blood from the penis, which at low doses may improve erectile function, but at the relatively higher dose of 80 μg pVAX-Vcsa1 can result in priapism.

### Discussion

These studies identify a gene, Vcsa1, that has changed expression in three models of erectile dysfunction and therefore may be a marker for organic ED. In addition, a physiological effect of the gene on erectile function was demonstrated. Gene transfer of a plasmid expressing the gene (pVAX-Vcsa1) seems to cause increased blood flow into the corpora in lower doses resulting in increased ICP/BP values in retired breeder animals after stimulation of the cavernous nerve, but causing priapism at higher doses.

Vcsa1 previously has been demonstrated to be downregulated in an animal model after ligation of the cavernous nerve (CN), which simulates nerve damage (neurogenic ED) (User et al. 2003). The present findings demonstrate that the Vcsa1 gene is downregulated in two other models of ED, a rat model for aging and STZ-diabetes. Although ED has overlapping pathophysiologies, the present findings demonstrate a common molecular change in three models of ED, therefore indicating that Vcsa1 should be a useful molecular marker for organic ED. It is possible that in this role it could act as a very early marker of ED since at one week, when there is a small, but not significant decrease in ICP/BP compared to nondiabetic controls (Table 1), there is approximately 50% decrease in the expression of Vcsa1 transcript in the corpora (FIG. 1). Because the regulation of Vcsa1 is under adrenergic control (Rosinski-Chupin et al. 2001), it is possible that the downregulation of Vcsa1 in ED associated with aging and diabetes is a result of CN neuropathy that occurs with both diabetes and aging (Bleustein et al. 2002).

A peptide product of the Vcsa1 gene has been shown to modulate the male rat's sexual behavior (Messaoudi et al. 2004). In the study of Messaoudi et al., treating rats with sialorphin revealed there was a significant stimulatory effect on the frequency of intromissions before ejaculation and on the propensity of males to engage in investigatory behavior directed to the female during the postejaculatory intervals. The studies by Messaoudi et al. did not directly address the effect of sialorphin on erectile capacity, and the rats were of an age and health where erectile capacity would not have demonstrated significant pathological features. In the studies described herein, it was possible to see the physiological effect of the Vcsa1 gene only in animals that had a reduced erectile function because of their age. In addition, the present experiments support the role of Vcsa1 as a prohormone. Gene transfer by intracorporal injection results in only a subset of corporal cells taking up the gene and expressing it. Therefore, an effect on the general physiology of the corpora suggests that the product of the gene affects more than a subset of genes.

The rat Vcsa1 gene shows 38% identity with a human homolog, submaxillary gland androgen regulated protein 3 homolog A precursor SMR3A (Isemura and Saitoh, 1997). The homology suggests that any role for Vcsa1 found in the rat may be performed by SMR3A in humans.

Overall, these studies indicate that Vcsa1 will be a useful marker for organic ED. It also demonstrates that Vcsa1 is involved in the regulation of blood circulation in the corpora. Because regulation of blood flow in corporal tissue is a key regulator of erectile function, this gene may also represent a novel therapeutic target for treating ED.

### Example II

### Sialorphin (The Mature Peptide Product of Vcsa1) Relaxes Corporal Smooth Muscle Tissue and Increases Erectile Function in the Aging Rat

### Materials and Methods

Experiments were carried out on 9- to 10-month-old retired breeder male Sprague-Dawley rats weighing more than 500 g (as described in Melman et al. 2005). When measurements were complete, animals were killed by placement within a CO₂ gas chamber. All protocols were approved by the Animal Use Committee and Internal Review board at the Albert Einstein College of Medicine.

The sialorphin peptide (2 mg) was dissolved in 0.5 ml of 0.01 N acetic acid and then was vortexed, and the solution was centrifuged for a few seconds at approximately 1000 g and 4° C. (to gather all the liquid at the bottom of the tube). The stock solution was stored in 25-μl aliquots (100 μg) at −70° C. Before use, the stock was thawed on ice, and phosphate buffered saline (pH 7.4) was added to bring the volume to 150 μl.

Intracorporal microinjection of sialorphin into rat corporal tissue was modified from previously described procedures, where plasmids were injected intracorporeally to investigate their effect on the ICP/BP ratio (Christ et al. 1998, 2004; Melman et al. 2005). The modifications from the previously described protocols were used in anticipation that as a mature peptide product, there would be a direct effect by sialorphin (compared with the gene transfer studies where a plasmid gene would have to be transcribed, translated, and processed before exerting an effect). Also as a hormonal peptide mediator, the sialorphin would be very susceptible to proteases or peptidases, reducing the time for it to exert its physiological effect. Therefore, the time from administration to cavernous nerve stimulation was reduced (to less than 1.5 h after administration) and animals were used as their own control (i.e., measurements were taken before and after treatment). Animals were anesthetized by an intraperitoneal injection of pentobarbital sodium (35 mg/kg), and the crus was exposed. A microinjection of 100 μg sialorphin in 150 μl of carrier solution (phosphate buffered saline, pH 7.4) was carried out. The other crus was isolated, and a 25-gauge needle was inserted to record intracavernous pressure. The cavernous nerves were identified adjacent to the prostate gland and electrostimulation performed with applied currents of 0.75 and 4 mA at 20 Hz and duration of 0.2 msec. The changes in ICP and systemic BP were recorded at both levels of neurostimulation. After two duplicate measurements, rats were given an intracorporal injection of 150 μl of 100 μg of sialorphin in carrier solution. After 20 minutes, ICP/BP measurements were initiated with applied currents of 0.75 and 4 mA. Mean ICP/BP (n=7) and standard deviation were calculated before and after treatment. Significant change in the ICP/BP value from that of the pretreatment group was determined by Student's t test.

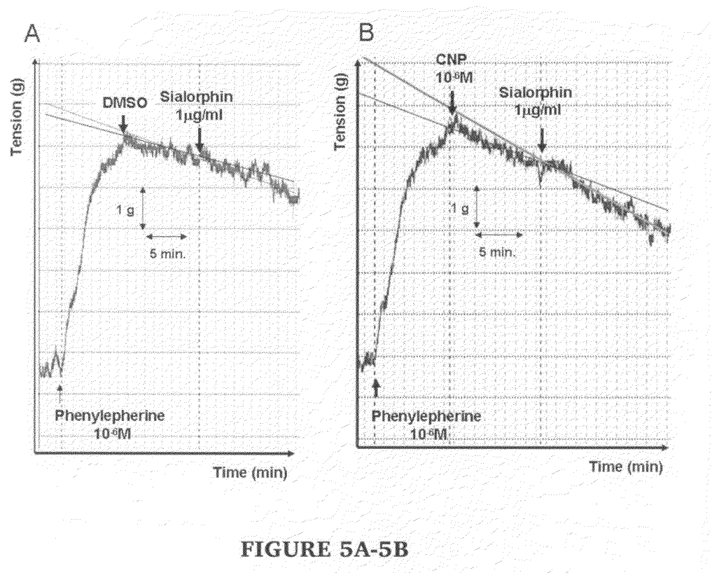

Studies of contractility of isolated smooth muscle tissue were carried out as described previously (Chang et al. 2003, Spektor et al. 2002). Four longitudinal strips of cavernous tissue from the crura of old animals (n=4) were dissected free from the tunica albuginea and were suspended between two small surgical hooks in a tension-measuring device (Multimyograph Model 610M, Copenhagen, Denmark) that allows simultaneous monitoring of four muscle strips. Tissue was equilibrated for 90 min in 6 ml Krebs-Henseleit buffer composed of 110 mM NaCl, 4.8 mM. KCl, 2.5 mM CaCl₂, 1.2 mM MgSO₄, 1.2 mM KH₂PO₄, 25 mM NaHCO₃, 11 mM glucose, and dextrose in glass-distilled water. Organ chambers were maintained at 37° C. and continuously bubbled with 95% O₂ and 5% CO₂ to maintain a mean pH of 7.4±0.1. Continuous recording of tension developed by the muscle strips was carried out using Powerlab software (Chart version 4.2.4, ADinstruments, CO) on a dedicated computer. Strips of corpus cavernosum first were precontracted with phenylephrine (10⁻⁶ M). Then, relaxation was induced using CNP (10⁻⁶ M). The change in the rate of relaxation after the addition of 1 μg/ml sialorphin was derived from the slope of the recording as the change in tension (g) over time (minutes) (as shown in FIG. 5). Results from four separate strips from four animals were averaged and the significance determined by the Student's t test.

### Results

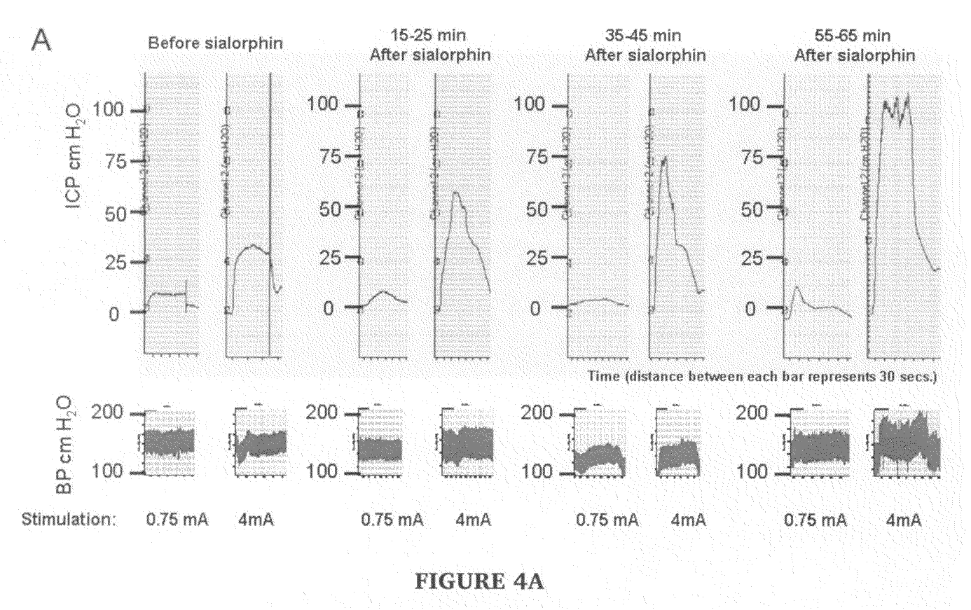

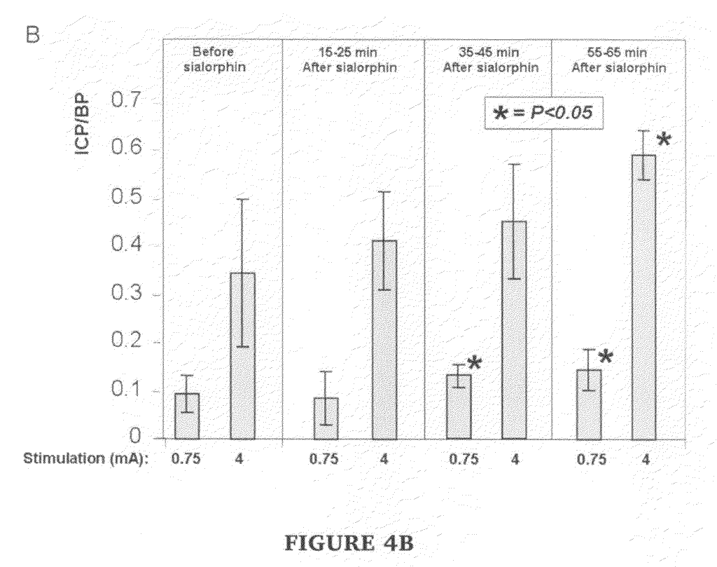

The ICP/BP ratio of animals was measured both before and immediately after the injection of 10 and 100 μg sialorphin, and then at 15 to 25, 35 to 45, and 55 to 65 min after the injection. There was no effect of sialorphin on the systemic blood pressure. The results of a representative experiment are shown in FIG. 4A, where ICP (upper panel) and BP (lower panel) were measured after electrostimulation at 0.75 and 4 mA before treatment with sialorphin and at various times after treatment. There seems to be a time-dependent significant increase in the effect of sialorphin on erectile function. This effect is noted at 35 to 45 and 55 to 65 min after intracorporal injection of sialorphin for the lower level of stimulation (0.75 mA), At 4 mA stimulation at the longest time studied after administration of sialorphin (55-65 min), there is a significant improvement of erectile function. The ICP/BP ratio approaches 0.6, which is considered a value at which an erection can be achieved. Carrier alone (n=3) and a lower dose of sialorphin (10 μg; n=3) had no significant effect on erectile function.

Studies of contractility of isolated smooth muscle tissue were performed as previously described (Chang et al. 2003, Spektor et al. 2002). Typical experiment results are shown in FIG. 3 and in Table 4. Tissue strips rapidly contracted on the addition of 1 μM phenylephrine to the organ bath media. Under the experimental conditions used in these experiments, the corpora strips slowly relax, taking more than 60 min to return to the baseline, at a rate of tension reduction of 0.052 g/min. However, on the addition of CNP (1 μM), there was a significant (67%) increase in the relaxation rate of the tissue. This rate of relaxation was increased further by addition of sialorphin (10 μg/ml), the protein product of Vcsa1, such that there was a 2.5-fold increase compared with the corporal strips treated with carrier alone. If sialorphin was incubated with the corporal strips without C-type natriuretic peptide (CNP), there was no significant effect on the relaxation rate, suggesting that sialorphin enhances the effect of CNP rather than the relaxation rate being affected independently.

TABLE 4
Sialorphin Can Increase the Relaxation of Isolated Corporal
Tissue Caused by C-Type Natriuretic Peptide

| | Rate of Tension |
|---|---|
| | Loss (g/min) |
| | (St. Dev.) |
| Carrier | 0.052 |
| (DMSO) | (0.017) |
| 1 μM CNP | 0.087* |
| | (0.012) |
| 1 μg/ml | 0.072 |
| Sialorphin | (0.018) |
| 1 μM CNP + | 0.13* |
| 1 μg/ml | (0.014) |
| Sialorphin | |

CNP = C-type natriuretic peptide;
DMSO = dimethyl sulfoxide.
The rate of tension loss was measured from four corporal smooth muscle strips from four different animals and averaged.
*A significant increase in rate of tension loss (p < 0.05, Student's t test) compared with strips treated with carrier alone.

### Discussion

Vcsa1 is downregulated in the corpora of diabetic and retired breeder rats with ED (see EXAMPLE I), as well as in the corpora of rats in a neurogenic model of ED (bilaterally ligated cavernous nerves) (User et al. 2003). Therefore, the expression of Vcsa1 can be a marker for organic ED. When the Vsca1 gene is introduced by intracorporal injection into aging rats, there was improved erectile function at lower doses, and priapism occurred as higher doses. These results demonstrate a direct involvement of Vcsa1 product in erectile function.

Penile erection is dependent on the relaxation of smooth muscle in the corpora cavernosum (Andersson 2001, 2003). It has been shown that endogenous selective 1-opioid receptor peptide agonists (endomorphins 1 and 2) can relax aortic vascular smooth muscle from the rat aorta by an endothelium-dependent mechanism (Hugghins et al. 2000). In addition, synthetic inhibitors of neutral endopeptidase (NEP) such as thiorphoran or phosphoramidon will enhance CNP-induced relaxation of porcine coronary artery (Marton et al. 2005). Vasopeptidase inhibitors are used to treat hypertension because of their ability to reduce vasocontraction and to enhance vasodilation (Lapointe and Rouleau, 2002). The mature peptide product of Vcsa1, sialorphin, displays potent analgesic activity in rats because of its ability to act as an NEP inhibitor, thereby activating 1- and 8-opioid receptor-dependent enkephalin pathways (Rougeot et al. 2003). Combining these observations, a reasonable hypothesis for the mechanism of action of gene transfer of Vcsa1 in restoring erectile function is that the gene product, sialorphin, acts locally as an inhibitor of NEP, thereby enhancing the activity of agonist-to-opioid receptors that stimulate smooth muscle relaxation. It is possible that NEP inhibitors at higher levels result in sustained smooth muscle relaxation so that there is activation of the pathways involved in priapism (Champion et al. 2005).

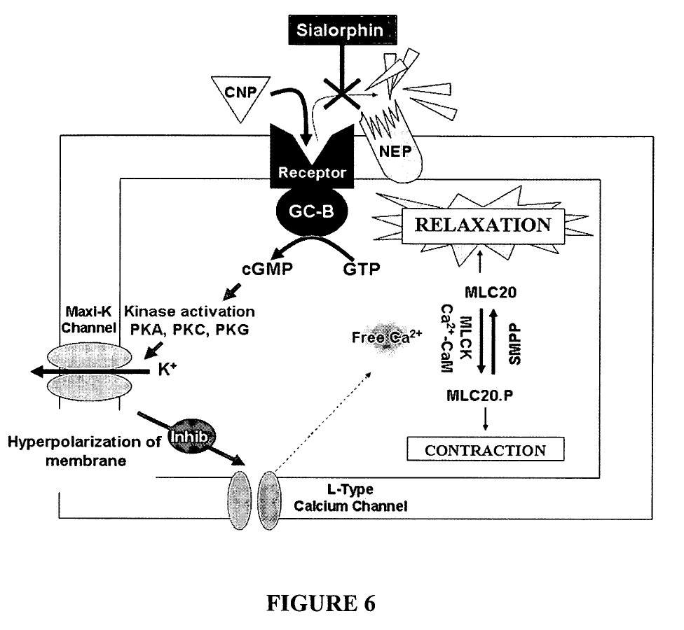

In addition to vasorelaxant effects, C-type natriuretic peptide (CNP) has also been shown to mediate hyperpolarizing effects. The hyperpolarizing events for CNP have been shown to be significantly diminished by iberiotoxin, a selective Maxi-K channel blocker (Otsuka et al. 2002). Further evidence for the involvement of potassium channels in natriuretic peptide-induced relaxation of smooth muscle cells is that high KCl potently suppressed relaxation (Otsuka et al. 2002). Gene transfer of plasmids expressing the Maxi-K channel (pVAX-hSlo) can be used to treat ED in aging animals (Melman et al. 2003). The potential of this therapy to treat human ED in clinical trials is being investigated (Melman et al. 2005). Activation or overexpression of the Maxi-K channels results in hyperpolarization of cells, which inhibits L-type calcium channels lowering intracellular calcium. Lowering intracellular calcium inactivates myosin light chain kinase, and then the myosin is dephosphorylated by smooth muscle myosin phosphatase, leading to smooth muscle relaxation (Hartshorne et al. 1998, Leblanc et al. 2005, Thomeloe and Nelson 2005) (FIG. 6).

CNP binds to corporal smooth muscle guanylyl cyclase B (GC-B) receptor present in both rats and rabbits, and it was demonstrated that CNP can cause relaxation of isolated rabbit corporal smooth muscle tissue (Kim et al. 1998, Kuthe et al. 2003). Therefore, the potential downstream mediators of sialorphin-induced relaxation of corporal smooth muscle cells may be through its action as an NEP inhibitor causing prolonged binding of CNP to corporal smooth muscle guanylyl cyclase B receptor present on the rat's smooth muscle tissue membrane, with consequent raising of the intracellular cGMP, causing activation of the Maxi-K channel. A proposed pathway by which sialorphin causes smooth muscle relaxation is shown in FIG. 6. Gene transfer protocols using the Maxi-K channel may have improved efficacy when combined with NEP inhibitors such as sialorphin.

Overall, these results indicate that gene transfer of a plasmid expressing Vcsa1 results in improved erectile function because of expression of its gene product, sialorphin. Furthermore, a component of ED observed with diabetes, aging and neuronal injury is likely associated with decreased production of sialorphin. Erectile function can be restored either with the administration of gene (Vscsa1) or its protein product (sialorphin). The results demonstrate that the role of sialorphin in erectile function is complex and indirect, being mediated through the inhibition of NEP, an enzyme that functions as a control mechanism to prevent sustained peptide-GC-B-receptor-cGMP activity. The limiting action of NEP is similar to that of phosphodiesterase (PDE) activity in the smooth muscle cells; it is enzyme that when blocked by PDE inhibitors also results in prolonged erection. The results of the present experiments demonstrate that injection of the sialorphin (the mature peptide product of Vcsa1) into the penile corpora significantly improves erectile function and suggests that inhibitors of NEP may provide novel targets for treatment of ED or may be useful for increasing the activity of existing treatments.

### Example III

### Human Homologue of Submandibular Rat 1 Gene (SMR1) (hSMR3A) as a Marker for Patients with Erectile Dysfunction

### Materials and Methods

Sequence Analysis and Comparison. The Basic Local Alignment Search Tool, available from the National Center of Biotechnology, National Institutes of Health, was used to search for gene and protein sequences with similarity to Vcsa1. Sequences were aligned using MultiAlin (Corpet, 1988), available on-line from Institut National de la Recherche Agronomique.

Cloning of hSMR3A and Construction of pVAX-hSMR3A. The full length gene was PCR amplified from human corporeal cell cDNA using the primers SMR3AF (5′-ggatgaaatcactgacttggatc-3′) (SEQ ID NO:7) and SMR3AR (5′-gtatttagggtgcaggagtaggg-3′) (SEQ ID NO:8), and hSMR3A was cloned into the pPCR-4-TOPO vector. After sequencing the insert to confirm the correct sequence, hSMR3A was subcloned into the pVAX vector (Invitrogen®) to create pVAX-hSMR3A.

Measurement of Intracorporeal Pressure/Blood Pressure (ICP/BP). A total of 17 Sprague-Dawley retired breeder rats at ages 9 to 10 months weighing greater than 500 gm were used to determine the effect of intracorporeal injection of pVAX-hSMR3A or the empty vector pVAX on erectile physiology. All study protocols were approved by the Animal Use Committee at the Albert Einstein College of Medicine.

For gene transfer experiments vectors/plasmids were microinjected into the rat corporeal tissue. The rats were anesthetized with pentobarbital sodium (35 mg/kg intraperitoneally). An incision was made through the perineum, the corpus spongiosum was identified and a window was made in the corpus spongiosum to identify the corpus cavernosum. Using an insulin syringe all microinjections consisted of a bolus injection of naked plasmid DNA into the corporeal tissue. The final volume of all microinjections was 150 μl.

For cavernosometry determining the ICP response to cavernous nerve (CN) stimulation, the rats were anesthetized with pentobarbital sodium (35 mg/kg intraperitoneally). An incision was made in the perineum and a window was made in the ischiocavernosus muscle to expose the corpus cavernosum. The CNs were identified adjacent to the prostate gland. The CN was directly electrostimulated with a delicate stainless steel bipolar hook electrode attached to a multijointed clamp. Each probe was 0.2 mm in diameter and the 2 poles were separated by 1 mm. Monophasic rectangular pulses were delivered by a signal generator that was custom made with a built-in constant current amplifier. Stimulation parameters were frequency 20 Hz, pulse width 0.22 milliseconds, duration 1 minute, and current 0.75 and 4 mA. Changes in ICP and systemic BP were recorded at each intensity of stimulation. Mean±SD ICP/BP and ANOVA were calculated for each treatment group. Significant differences between treatment groups were determined by Student's t test.

Patient Samples. Human corporeal tissue was procured from several patients during penile prosthetic implant surgery according to protocols approved by the AECOM/Montefiore Hospital Internal Review Board. Table 5 lists the conditions and ages of patients 1 to 10. Tissue samples were immediately flash frozen after removal in liquid nitrogen and stored at −70° C. until RNA and cDNA preparation.

TABLE 5
Patient information.

| | Failure | | | | | | | |
|---|---|---|---|---|---|---|---|---|
| | | Intra- | | | | | Previous | |
| Pt # | Phospho- | cavernous | | Diabetes | | | Pelvic | |
| Age | diesterase-5 | Injection | MUSE ® | Mellitis | Other Disease | Smoker | Surgery | Prosthesis |
| 0A | No | No | No | | | | | |
| 26 | | | | | | | | |
| 0B | No | No | No | | | | | |
| 35 | | | | | | | | |
| 0C | No | No | No | | | | | |
| 51 | | | | | | | | |
| 1 | Yes | No | No | Yes | Hypertension | No | Radical | Semirigid |
| 62 | | | | | | | retropubic | |
| | | | | | | | prostate- | |
| | | | | | | | ectomy + | |
| | | | | | | | colon | |
| 2 | No | No | No | Yes | Hypertension, | No | No | Semirigid |
| 66 | | | | | congestive | | | |
| | | | | | heart failure, | | | |
| | | | | | hyper- | | | |
| | | | | | cholesterol | | | |
| 3 | Yes | No | No | Yes | Benign | No | Prostate | Inflatable |
| 64 | | | | | prostatic | | Cabrachy- | |
| | | | | | hyperplasia, | | therapy | |
| | | | | | gastro- | | | |
| | | | | | esophageal | | | |
| | | | | | reflux disease | | | |
| 4 | No | No | No | Yes | Hypertension, | No | No | Semirigid |
| 65 | | | | | peripheral | | | |
| | | | | | vascular | | | |
| | | | | | disease | | | |
| 5 | Yes | Yes | Yes | Yes | No | No | No | Semirigid |
| 68 | | | | | | | | |
| 6 | No | No | No | No | Peripheral | Yes | No | Inflatable |
| 72 | | | | | vascular | | | |
| | | | | | disease, | | | |
| | | | | | osteoporosis | | | |
| 7 | No | No | No | No | Hyper- | Yes | No | Inflatable |
| 45 | | | | | cholesterol | | | |
| 8 | No | No | No | No | Hypo- | Yes | No | Inflatable |
| 79 | | | | | thyroidism | | | |
| 9 | No | No | No | No | Gout, no | No | Penile re- | Inflatable |
| 72 | | | | | family history | | vascularization | |
| 10 | Yes | No | No | No | Prostate | No | Laparoscopic | Inflatable |
| 59 | | | | | Cancer | | radical | |
| | | | | | | | prostate- | |
| | | | | | | | ectomy | |

Automobile accident, sex change surgery and penile cancer surgery in patients 0A, 0B and 0C, respectively.

Isolation of Patient RNA and Quantitative RT-PCR. Total RNA was extracted from frozen tissue with TRIzol® according to manufacturer instructions. Briefly, approximately 50 mg tissue were added to 1 ml TRIzol reagent and homogenized using a Polytron™ homogenizer for 30 seconds. Homogenized tissues were incubated for 5 minutes at room temperature, followed by the addition of 200 μl chloroform. After mixing, the aqueous phases were separated by centrifugation at 12,000× gravity for 15 minutes at 4° C. and they were then transferred to a clean tube. RNA was precipitated from the aqueous phase by the addition of isopropyl alcohol and pelleted by centrifugation at 12,000× gravity for 15 minutes at 4° C., washed once with 75% ethanol and again pelleted at 12,000× gravity for 15 minutes. Ethanol was aspirated and the RNA pellet was dried and then dissolved in sterile water.

Total RNA (1 μg) was reverse transcribed to first strand cDNA primed with oligo (deoxythymidine) using the Superscrip® First-Strand Synthesis System for real-time polymerase chain reaction (PCR). RNA was denatured for 5 minutes at 65° C. and immediately cooled on ice. RNA was then combined with Superscript II RT, 40 U RNaseOUT™ recombinant ribonuclease inhibitor and reverse transcriptase (RT) reaction buffer. cDNA synthesis was then performed for 50 minutes at 42° C. RT products were amplified using SYBR® Green 2×PCR Master Mix. Real-time quantitative PCR analysis was performed using a 7300 real-time PCR system (Applied Biosystems®). The primers for hSMR3A were forward 5′-CTATGGTCCAGGGAGATTTCC-3′ (SEQ ID NO:9) and reverse 5′-GAGGAGGAAGAGAGTGTGATTG-3′ (SEQ ID NO:10). GAPDH (forward primer 5′-GCCGCCTGCTTCACCACCTTCT-3′ (SEQ ID NO:5) and reverse primer 5′-GCATGGCCTTCCGTGTTCCTACC-3′) (SEQ ID NO:6) served as an endogenous control. PCR reactions for all samples were performed in 96-well plates with 1 μl cDNA, 100 nM of each primer and 12.5 μl SYBR® Green in a 25 μl reaction volume. Cycling conditions were SYBR Green DNA polymerase activation at 95° C. for 10 minutes, 40 cycles of denaturation at 95° C. for 15 seconds and annealing/extension at 60° C. for 1 minute. Real-time PCR results are presented as threshold cycles normalized to that of the GAPDH gene. The relative quantified value for each target gene is expressed as 2^{-(Ct-Cc)}, where crossing threshold (Ct) and Cc represent mean threshold cycle differences after normalizing to GAPDH. Transcript expression was analyzed using the comparative Ct method, also known as the 2^{-δδCt} method. This method was applicable because the efficiency of the SMR3A primers for generating products was found to be close to that of the housekeeping gene glyceraldehyde-3-phosphate dehydrogenase (GAPDH), which was used to normalize samples.

### Results

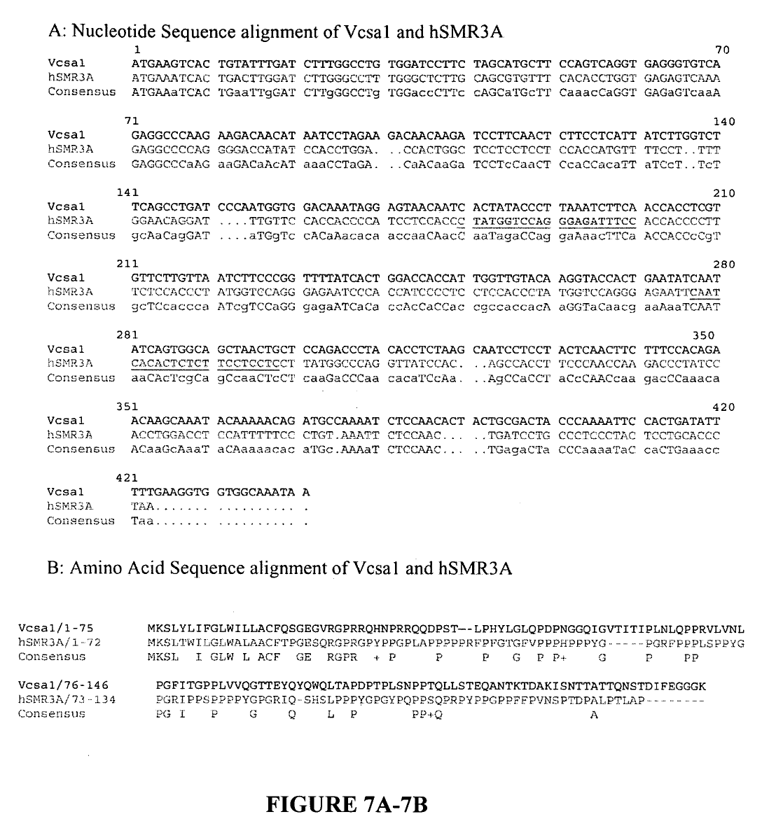

DNA and Sequence Analysis. GenBank® was searched for human proteins with the greatest similarity to Vcsa1. The closest human gene with homology to Vcsa1 was identified as hSMR3A, which has 34% identity with Vcsa1 at the protein level (FIG. 7). In the first 38 amino acids of the protein, which encodes the functional mature peptide sialorphin, there is 55% identity. This level of identity suggests that the proteins perform similar physiological roles.

Effect of Intracorporeal pVAX-hSMR3A Injection Into Retired Breeders on Erectile Physiology. pVAX-Vcsa1 injection into retired breeder rats can improve erectile physiology when 25 μg are injected intracorporeally, but higher amounts of plasmid results in priapism. To confirm that hSMR3A is a functional homologue of Vcsa1, these experiments were repeated to determine if hSMR3A has comparable physiological effects on the penis.

When 25 μg pVAX-hSMR3A were intracorporeally injected into retired breeder rats, there was significant improvement in the erectile response, as indicated by an increased ICP-to-BP ratio compared with that in control rats treated with the empty vector pVAX (Table 6). The values obtained were similar to those in experiments in which the effect of gene transfer of pVAX-Vcsa1 into the corpora of retired breeders was investigated for an effect on erectile function.

TABLE 6
ICP/BP measurements in retired breeder rats after pVAX or pVAX-
hSMR3A gene transfer and cavernous nerve (CN) electrostimulation.

| Intracorporeal | | Mean ± SD ICP/BP | | |
|---|---|---|---|---|
| Injection (dose) | No. Rats | Baseline | 0.75 mA | 4 mA |
| Control pVAX | 8 | 0.063 ± 0.02 | 0.22 ± 0.12 | 0.33 ± 0.06 |
| (100 μg) | | | | |
| pVAX-hSMR3A: | | | | |
| 25 μg | 3 | 0.153 ± 0.06 | 0.28 ± 0.07 | 0.61 ± 0.02 |
| 100 μg | 5 | 0.102 ± 0.05 | 0.13 ± 0.07 | 0.39 ± 0.01 |

Significantly different vs control (Student's t test p < 0.05).

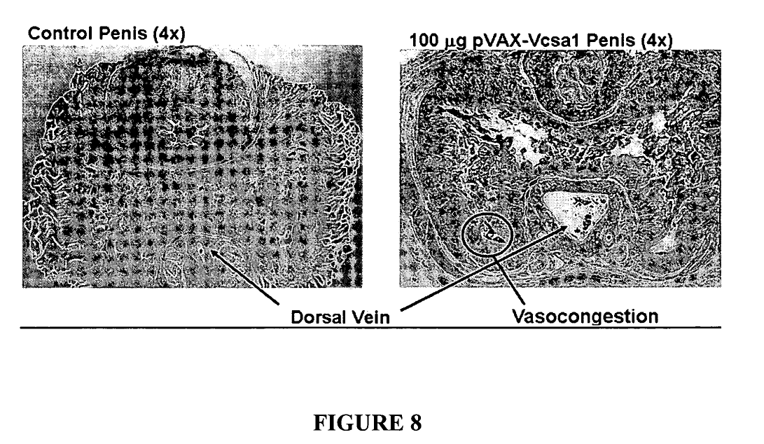

Also similar to experiments for Vcsa1, higher doses of the pVAX-hSMR3A plasmid resulted in only slight improvement in ICP-to-BP ratios (Table 6), although there was visible and histological evidence of a priapitic episode. The histological appearance of 4 of the 5 animals treated with pVAX-hSMR3A showed visible indications of edema, which is a possible indication of a vasocongested state, whereas in untreated control animals corporeal morphology appeared normal. Histological examination and comparison to control animals also suggested that SMR3A causes changes in penile morphology, which might have been a result of the vasocongested (priapism-like) state (FIG. 8). The dorsal vein was greatly enlarged. This would occur if there was increased blood flow or post-penile obstruction, which was not observed. In addition, there was evidence of sinusoidal congestion of blood in animals treated with hSMR3A but not in control animals. Overall the occurrence of vasocongestion (a priapism-like state) has not been observed in the history of animal experiments at the inventors' department in which vasodilating drugs or genes were injected into the corpora.

The sequence and functional similarity of Vcsa1 to hSMR3A suggests that they are indeed the homologues of each other. An analysis of human corporeal samples was performed to determine if hSMR3A is present in patients with no reported ED and down-regulated in patients with ED.

Detection of SMR3A in Human Corpora. Although the rat Vcsa1 gene was originally isolated from the rat submandibular gland (hence, the original designation SMR1) and is highly expressed in that tissue, it appears to be expressed in various other tissues (Isemura et al. 2004), including rat corpus cavernosum tissue. Therefore, it was determined if hSMR3A is similarly present in human corporeal tissue. Corporeal samples were available from patients 0A, 0B and 0C, who did not report ED (Table 5). In these 3 patients hSMR3A was clearly detectable using quantitative RT-PCR, demonstrating that the gene is expressed in human corporeal tissues.

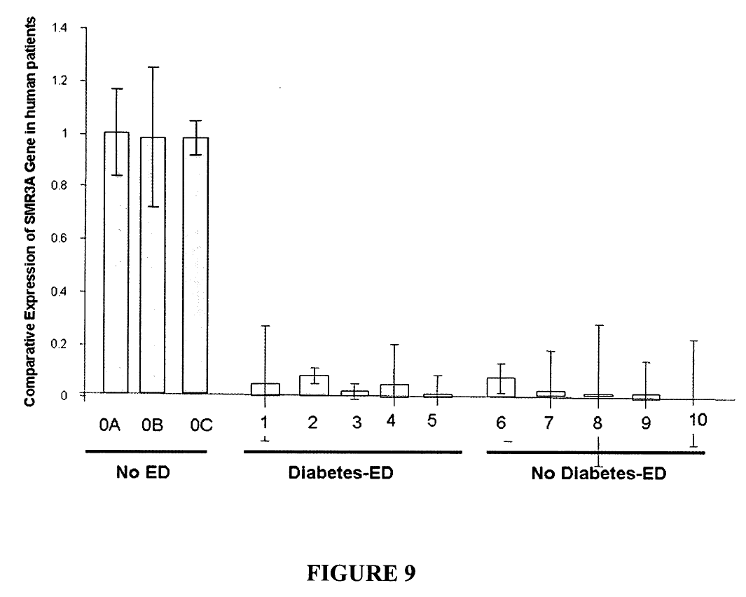

Decreased hSMR3A Expression in Patients With ED Compared to That in Patients Without ED. hSMR3A levels were determined in patients undergoing prosthetic implant surgery (Table 5 and FIG. 9). hSMR3A expression in the patients was normalized to glyceraldehyde-3-phosphate dehydrogenase (GAPDH) and the expression level was compared to that in patient 0A without ED. In patients with ED there were significantly lower levels of expression compared to those in the 3 control patients (more than a 10-fold decrease, Student's t test p<0.5), suggesting that, as in the rat model, hSMR3A is a marker for erectile function. The ED patients were grouped into those with and without diabetes. The two groups had significantly lower levels of hSMR3A expression compared to control patients (Student's t test p<0.5). However, compared to the control mean age of 37 years the median age in the 2 ED groups with and without diabetes was higher (each mean 65). Therefore, in these groups of patients it was not possible to distinguish if the reported ED was a result of diabetes or age. However, overall down-regulation of hSMR3A expression is a marker for ED caused by several factors.

### Discussion

Evidence is provided herein that hSMR3A is the human homologue of the Vcsa1 gene. This conclusion is based on sequence comparison and gene transfer of hSMR3A by intracorporeal injection into an aging rat model. Similar to Vcsa1, hSMR3A can improve erectile function when 25 pg are intracorporeally injected and it can cause priapism at higher amounts. hSMR3A is expressed in human corpora tissue and is down-regulated in patients with ED. Down-regulation of hSMR3A is highly significant despite the small number of control patient samples that could be obtained for this study. The current study indicates that hSMR3A acts as a marker for human ED.

Although in the rat Vcsa1 and hSMR3A appear to have a direct role in erectile function since intracorporeal injection of plasmids expressing the gene can improve erectile function in an aging model of ED, down-regulation of the gene could be a cause or an effect of ED. In the rat Vcsa1 gene expression is regulated by androgens, which can cause 100 to 200-fold enhancement of Vcsa1 in the acinar cells of rat submandibular glands during puberty (Rosinski-Chupin et al 1988, 2001). It remains to be determined if the regulation of hSMR3A expression is also under hormonal regulation.

The mature peptide product of Vcsa1, sialorphin, is able to directly improve erectile function in the aging rat. Sialorphin acts as an inhibitor of rat membrane bound neutral endopeptidase (NEP) (Rougeot et al. 2003). The ability of sialorphin to prolong the activity of agonists that are normally broken down by NEPs may cause heightened smooth muscle relaxation in the corpora cavernosa, leading to penile erection. Given the similarity of the amino acid sequence and the fact that SMR3A and Vcsa1 are down-regulated with ED, it is likely that hSMR3A also gives rise to peptide products that act as NEP inhibitors and, thereby, cause human corporeal smooth muscle tissue relaxation. Recently PROL1, another member of the Vcsa1 gene family found in humans, was shown to give rise to a protein product called opiorphin, which is secreted in saliva. This protein also acts as an NEP inhibitor, suggesting that the physiological effect of this family of proteins is mediated through NEP inhibition (Wisner et al. 2006).

The identification of hSMR3A as a marker for ED has applications as a diagnostic tool for organic ED and in the development of novel therapies. In the era of agents that are noninvasive and successful for treating ED, the quest to establish an etiological diagnosis has been downplayed. However, the potential ability to suggest to the patient that the condition is reversible, i.e. psychogenic if the level is normal, with an accurate but invasive test (diagnostic corporeal biopsy) or with the development of a noninvasive immunoassay would be of significance to the physician and patient, particularly young men who are convinced that they have a nonreversible physical problem, as well as for reimbursement issues for therapy by insurance companies (Melman et al. 2006). In addition, it is increasingly recognized that ED is an important marker of vascular disease and there is growing evidence that ED and cardiovascular disease share common mechanisms of development through vascular endothelial dysfunction (Muller and Mulhall, 2006). Indeed, it has been recommended that patients with ED should be investigated for cardiovascular disease (Thompson et al. 2005). The development of a test for organic ED in men who approach physicians for treatment represents an enormous potential for prescreening and the prevention of more serious vascular complications. Given the studies demonstrating that gene transfer of the Vcsa1 gene and its protein product in rats can restore erectile function, these results suggest that therapies that increase the expression of the hSMR3A gene or other genes in the Vcsa1 gene family, resulting in peptide products that act as NEP inhibitors, could have a positive impact on human erectile function.

In summary, these results indicate that hSMR3A can act as a marker for erectile dysfunction associated with diabetic and nondiabetic etiologies. Given the studies demonstrating that gene transfer of the Vcsa1 gene and intracorporeal injection of its protein product in rats can restore erectile function, these results suggest that therapies that increase the hSMR3A gene and product expression could have a positive impact on erectile function.

### Example IV

### PROL1 is Down-Regulated in Human Erectile Dysfunction

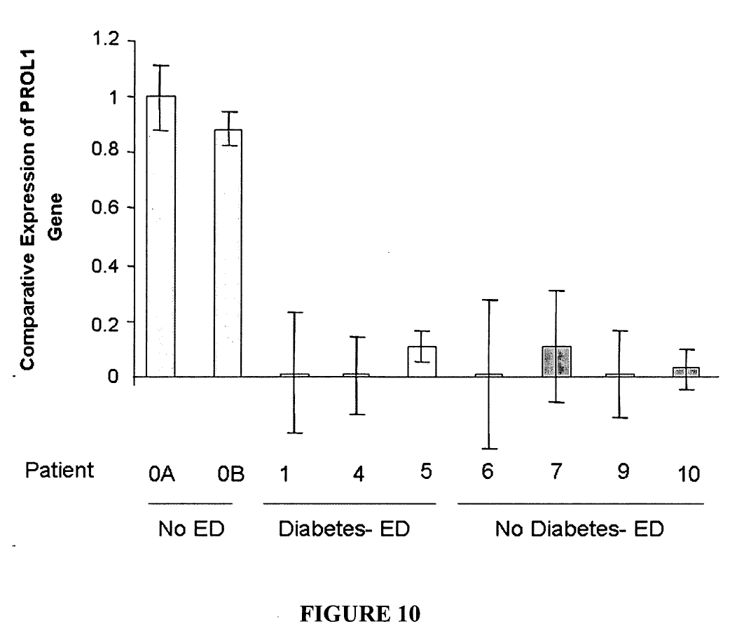

Expression of Prol1 transcripts was analyzed using the comparative crossing threshold (Ct) method (also known as the 2-{delta}{delta}Ct method). This method was applicable because the efficiency of the Prol1 primers (Forward Primer: GCT CTT ATT TCA TGT TTC ACA CCC AG (SEQ ID NO:11): Reverse Primer: TAA CCC GGA AAG AGT CGA GGT (SEQ ID NO:12)) in generating products was found to be close to that of the house keeping gene, GAPDH, which was used to normalize samples. Level of expression is compared to Patient 0A. The patients are the same as described in Table 5. As shown in FIG. 10, Prol1 is down-regulated in subjects with ED, both in ED subjects with diabetes and in ED subjects without diabetes.

### Example V

### Correlation of Vcsa1 Expression with Gene Therapy and Pharmacological Treatment that Restore Erectile Function

Vcsa1 gene is down-regulated in three models of erectile dysfunction (ED); namely diabetic, age-related and neurogenic (bilaterally-ligated cavernous nerve) models of ED. In order to determine if Vcsa1 expression is a useful marker for treatments that restore erectile function, Vcsa1 was assessed in connection with the following treatments: gene transfer, pharmacotherapy using tadalafil (Cialis®), and a combination of both treatments. Gene transfer of pVAX-hSlo, which expresses the alpha-subunit of the Maxi-K gene, restores erectile function in aging and diabetic rats (Christ et al. 1998, 2004; Melman et al. 2003) and is presently undergoing clinical trials in humans (Melman et al. 2005). Cialis® is presently one of the FDA approved PDE5 inhibitors used for treating patients with ED (Moore et al. 2005).

### Materials and Methods

Treatment of Retired Breeders. Retired breeder, male Sprague Dawley rats, 9-10 months old and weighing >500 g were used in these studies (from Charles River Breeding Laboratories, Wilmington, Mass., USA). They are commonly employed animal models for age-related ED (Christ et al. 1998; Melman et al. 2003). All animal protocols are approved by the Animal Use Committee at the Albert Einstein College of Medicine, Bronx, N.Y.

Microinjection of pVAX, pVAX-hSlo and pSMAA-hSlo into rat corporal tissue was performed essentially as previously described (Christ et al. 1998; Melman et al. 2003, 2008). Briefly, animals were anesthetized by an intraperitoneal injection of pentobarbital sodium (35 mg/kg). An incision was made in the perineum and a window was made in the ischiocavernous muscle to expose a penile crus. All microinjections consisted of a single bolus injection of 100 μg of plasmid DNA into the corporal tissue using an insulin syringe. The final volume of all microinjections was 150 μl of phosphate buffered saline (PBS) and sucrose (20%). One week after intracorporal injection the intracavernosal pressure/systemic blood pressure (ICP/BP) response to electrostimulation of the cavernous nerve was determined.

In a second set of animals (retired breeder, male Sprague Dawley rats) in which the effect of tadalafil was to be measured alone or in combination with pVAX-hSlo, 1000 μg pVAX-hSlo was administered to 10 animals 1 month prior to determination of erectile function and gene expression. Five of these animals were treated with 2.5 mg/kg of tadalafil orally two hours prior to ICP/BP measurement. A third group of five animals were untreated controls, and the fourth group of five animals treated with 2.5 mg/kg of tadalafil orally. two hours prior to ICP/BP measurement.

ICP/BP measurement. ICP/BP was measured essentially as previously described (Christ et al. 1998; Melman et al. 2003). Briefly, animals were anesthetized by intraperitoneal injection of pentobarbital sodium (35 mg/kg). An incision was made in the perineum, and the corpus cavernosum exposed. The cavernous nerves were identified adjacent to the prostate gland. Direct electrostimulation of a cavernous nerve was performed with a delicate stainless steel bipolar hook electrode attached to the multi-jointed clamp. Each probe was 0.2 mm in diameter; the two poles were separated by 1 mm. Prior to electrostimulation the longest visually observed erection was measured. Monophasic rectangular pulses were delivered by a signal generator (custom-made and with built-in constant current amplifier). Stimulation parameters were as follows: frequency, 20 Hz; pulse width, 0.22 ms; duration, 1 min. Current was applied at 0.75 and 4 mA. The changes in ICP and systemic BP were recorded at each level of neurostimulation. The mean ICP/BP, standard deviation, and analysis of variance were calculated for each of the treatment groups.

RNA Isolation from corporal tissue. Following the ICP/BP measurement, animals were euthanized and corporal tissues isolated and flash frozen in liquid nitrogen. Total RNA was extracted from frozen tissue with TRIzol. Briefly, approximately 50 mg tissue was added to 1 ml TRIzol reagent and homogenized using a polytron homogenizer (Brinkman, Westbury, N.Y.) for 30s. The homogenized tissues were incubated for 5 min at room temperature followed by addition of 200 μl of chloroform. After mixing, the aqueous phases were separated by centrifugation (12000×g for 15 min) at 4° C. and then were transferred to a clean tube. The RNA was precipitated from the aqueous phase by addition of isopropyl alcohol and pelleted by centrifugation at 12000×g for 15 min at 4° C., washed once with 75% ethanol, and again pelleted at 12000×g for 15 min. The ethanol was aspirated and the RNA pellet was dissolved in sterile water.

Gene expression by microarray analysis. The RNA was used to perform microarray analysis of global gene expression using the RGU-230A affymetrix microarray. Quality control of the RNA, labeling and hybridization to the microarray were performed by standard Affymetrix protocols by the Albert Einstein College of Medicine Affymetrix Facility. A total of 12 microarray analyses were performed using 4 chips each for the control, pVAX-hSlo and pSMAA-hSlo treated animals. Gene expression in the treated animals was compared with controls using AffylmGUI software, available from www.bioconductor.org. Over-represented ontological themes were identified using the DAVID database (Database for Annotation, Visualization and Integrated Discovery (http://david.abcc.ncifcrf.gov/homejsp).

Transfection of rat corporal smooth muscle cells with pVAX-hSlo and pSMAA-hSlo. Rat corporal smooth muscle cells were isolated essentially as described by Jackson et al. (1996). Briefly, corporal tissue was cut into small pieces followed by dissociation by incubation with 30 U/ml papain and 1 mg/ml dithioerythritol for 35 min followed by incubation with 1 mg/ml collagenase, 2.5 U/ml elastase, and 1 mg/ml soybean trypsin inhibitor for 25 min in a solution containing 137 mM NaCl, 5.6 mM KCl, 1 mM MgCl₂, 0.42 mM Na₂HPO₄, 0.44 mM NaH₂PO₄, 4.2 mM NaHCO₃, 10 mM Hepes and 1 mg/ml albumin at 37° C.

Cells were grown in low glucose (1 g/L) DMEM and 10% FBS. Cells at passage 1 or 2 were transfected in 10 cm cell culture dishes with Fugene HD transfection reagent (Roche Applied Science, Indianapolis, Ill.) at about 70% confluence according to the instructions of the manufacturer, with a transfection reagent/DNA ratio of 3:2 (μl/μg). 48 h following transfection cells were harvested and RNA extracted with RNeasy mini kit (Qiagen, Valencia, Calif.). RNA was used for microarray analysis or for quantitative RT-PCR. The number of sample replicates for each experiment are described in the figure legends.

Measurement of gene expression by quantitative RT-PCR. RNA extracted either from corporal tissue or transfected corporal smooth muscle cells was used to analyze gene expression by quantitative RT-PCR. One microgram total RNA was reverse-transcribed to first-strand cDNA primed with Oligo(dT) using the Superscript (Invitrogen) First-Strand Synthesis System for real-time PCR. RNA was denatured for 5 min at 65° C. and immediately cooled on ice. Then RNA was combined with the Superscript II RT, 40 units of RNaseOUT recombinant ribonuclease inhibitor, and RT reaction buffer. cDNA synthesis was performed for 50 min at 42° C. RT products then were amplified using Sybr Green 2×PCR Master Mix (PE Applied Biosystems, Warrington, UK). Real-time quantitative PCR analysis was performed using the 7300 real-time PCR system (Applied Biosystems, Foster City, Calif.). The primers used to quantify expression levels of the genes are shown in Table 7.

TABLE 7

Primers used to confirm microarray gene expression

| | |
|---|---|
| Gene | Primer |

| | | | |
|---|---|---|---|
| Vcsal | forward primer | 5′-GAGGGTGTCAGAGGCCC-3′ | (SEQ ID NO: 3) |
| | reverse primer | 5′-GAGCAGTTAGCTGCCACTGATA-3′ | (SEQ ID NO: 4) |
| Slo | forward primer | 5′-TACTTCAATGACAATATCCTCACCCT-3′ | (SEQ ID NO: 13) |
| | reverse primer | 5′-ACCATAACAACCACCATCCCCTAAG-3′ | (SEQ ID NO: 14) |
| Expi | foward primer | 5′-TGTTCCAATGGCTGTGGTCA-3′ | (SEQ ID NO: 27) |
| | reverse primer | 5′-GGCCATCAGTCGTGCTTATGA-3′ | (SEQ ID NO: 28) |
| Krt1-18 | forward primer | 5′-CAGACCTTGGAGATTGACCTGG-3′ | (SEQ ID NO: 29) |
| | reverse primer | 5′-TTGCTCCATCTGCACCCTGTA-3′ | (SEQ ID NO: 30) |
| Cav | forward primer | 5′-ACCATCTTCGGCATCCCTATG-3′ | (SEQ ID NO: 31) |
| | reverse primer | 5′-AGGAAGCTCTTGATGCACGGT-3′ | (SEQ ID NO: 32) |
| Eef1a1 | forward primer | 5′-GTCAGAACGCAGGTGTTGTGAA-3′ | (SEQ ID NO: 33) |
| | reverse primer | 5′-GCCGGAATCTACGTGTCCAAT-3′ | (SEQ ID NO: 34) |
| Emp1 | forward primer | 5′-TCAAAGTGCATGCCCACCA-3′ | (SEQ ID NO: 35) |
| | reverse primer | 5′-GCGATGGAACATGTGCATCTC-3′ | (SEQ ID NO: 36) |
| RGD: 1303126 | forward primer | 5′-TCTGACGGCAGGTCCTATGAGT-3′ | (SEQ ID NO: 37) |
| | reverse primer | 5′-TGGCCAGCATCTTTGCATC-3′ | (SEQ ID NO: 38) |
| Muc10 | forward primer | 5′-TCCCACCAAGGAGCAACATTAA-3′ | (SEQ ID NO: 39) |
| | reverse primer | 5′-GGATGTGGTTTTGGCTGGAAG-3′ | (SEQ ID NO: 40) |
| Alas2 | forward primer | 5′-ACCTCCCCTGCTGATTCAGAAT-3′ | (SEQ ID NO: 41) |
| | reverse primer | 5′-ACGGTATGTGTGGTCCTGCTTC-3′ | (SEQ ID NO: 42) |
| S100a9 | forward primer | 5′-ACCCTGAACAAGGCGGAATT-3′ | (SEQ ID NO: 43) |
| | reverse primer | 5′-TTTGTGTCCAGGTCCTCCATG-3′ | (SEQ ID NO: 44) |
| Pbsn | forward primer | 5′-TGCTCACACTGGATGTGCTAGG-3′ | (SEQ ID NO: 45) |
| | reverse primer | 5′-TCCACGCTACTGGCAGCTAAGT-3′ | (SEQ ID NO: 46) |
| Rp124 | forward primer | 5′-TCGAGCTGTGCAGTTTTAGTGG-3′ | (SEQ ID NO: 47) |
| | reverse primer | 5′-GCGGACTCACATTTGGCATTA-3′ | (SEQ ID NO: 48) |
| GAPDH | forward primer | 5′-GCCGCCTGCTTCACCACCTTCT-3′ | (SEQ ID NO: 5) |
| | Reverse primer | 5′-GCATGGCCTTCCGTGTTCCTACC-3′ | (SEQ ID NO: 6) |

The PCR reactions for all samples were performed in 96-well plates, with 2 μl cDNA, 100 nM each primer, and 12.5 μl of Sybr Green in a 25-μl reaction volume. The cycling conditions were as follows: activation of Sybr Green DNA polymerase at 95° C. for 10 min, 40 cycles of denaturation at 95° C. for 15 s, annealing/extension at 60° C. for 1 min. Results from real-time PCR were presented as threshold cycles normalized to that of the RPL24 gene or GAPDH. The relative quantified value for each target gene in corpora of treated rats is expressed as 2^{-(Ct-Cc)} (Ct and Cc are the mean threshold cycle differences after normalizing to RPL24). Expression of transcripts was analyzed using comparative crossing threshold (Cₜ) method (also known as the 2^{-{delta}{delta}Ct} method). This method was applicable because the efficiency of the primers in generating products was found to be close to that of the house keeping gene which was used to normalize samples.

### Results

Treating aging rats with pVAX-hSlo or pSMAA-hSlo restores erectile function. The ability of intracavernosal injection of pVAX-hSlo, in which expression of hSlo is driven by the cytomegalovirus (CMV) promoter, to normalize the observed ED that occurs due to aging in retired breeder rats has been previously reported (Melman et al. 2003). A plasmid, in which hSlo was expressed from a smooth muscle specific promoter (the smooth muscle alpha actin (SMAA) promoter), called pSMAA-hSlo, gave similar results (Melman et al. 2008). In the present study the effect of these two gene transfer treatments were compared to an empty backbone vector (pVAX) to improve erectile function in retired breeder animals and simultaneously measure differences in gene expression.

Three groups of five animals were given intracorporal injections of 100 μg of the different plasmids (pVAX-hSlo, pSMAA-hSlo and pVAX). Animals treated with either plasmid expressing hSlo showed a significant improvement in erectile function, as determined by the ICP/BP ratio following stimulation of the cavernous nerve. The pVAX treated animals showed no normalization of erectile function.

Identification of changes in gene expression levels by microarray analysis. Having confirmed that the gene transfer was effective in increasing erectile function, corporal tissue was harvested from all animals and RNA was extracted and used to compare the gene expression patterns in the three groups of animals. Four microarray chips were analyzed for each group of rats. The gene expression in animals treated with plasmid expressing hSlo was compared to animals treated with the plasmid backbone (pVAX, control).

In corpora treated with pVAX-hSlo, a total of 144 genes show >1.5-fold change in expression level compared to the pVAX control treated group, whereas animals treated with pSMAA-hSlo had a total of 189 genes with >1.5-fold level of change. However, there was a considerable overlap in the genes changed in expression. This suggests that treatment with either plasmid expressing hSlo triggers similar analogue physiological and molecular effects when administered in vivo. Overall the changed genes represent less than 1% of the total genes (˜31,100) on the chip.

To further investigate the significance of the gene changes, the entire list of up- and down-regulated genes was sorted by ontological themes using the DAVID database (Database for Annotation, Visualization and Integrated Discovery (http://david.abcc.ncifcrf.gov/homejsp). DAVID provides a comprehensive set of functional annotation tools to understand biological meaning behind large list of genes. Analysis of the entire list of up-regulated genes after treatment of animals with pVAX-hSlo and pSMAA-hSlo indicated that the intermediate filament group of genes, the keratins, are up-regulated as a significant ontological theme. In the same way, the down-regulated genes were analyzed for ontological significance using the DAVID database. Both pVAX-hSlo and pSMAA-hSlo treatments caused down-regulation of an ontological group of genes involved in transcription regulation, though some of the genes in the same group did not correlate.

Ontological grouping does not consider genes that may be functionally relevant but have unknown function and have not been assigned to an ontological category. Therefore, the 20 most up- and down-regulated genes were looked at in detail. Several genes normally associated with the submandibular gland (Muc10, coding for the submandibular gland salivary protein mucin 10, RGD: 708577, coding for the common salivary protein 1 and Vcsa1, coding for the variable coding sequence protein Al) are amongst the most up-regulated genes in the case of both pVAX-hSlo and pSMAA-hSlo gene therapies. The microarray analysis did not show any significant change in the expression of the Slo gene. Quantitative-RT-PCR analysis with primers that would detect all Slo transcripts (endogenous rat Slo and plasmid derived hSlo) confirmed that there was no significant change in the overall expression of the gene in the corpora (See Table 8). It is known that there is only localized uptake of intracorporally injected plasmid into relatively small numbers of cells (Christ et al. 1998), so that when hSlo expression in the whole corpora is measured it might be expected that hSlo expression would not be greatly effected. Indeed, this result confirms past studies which have shown that despite a positive physiological effect of plasmids expressing the hSlo gene on erectile function, after one week there is no effect on total Slo levels expressed (Christ et al. 1998; Melman et al. 2003, 2008).

In order to confirm changes in expression detected by microarray, the same RNA used for microarray analysis was analyzed by quantitative-RT-PCR using primers against select genes (Table 8). The ribosomal protein 24 gene (RPL24) was selected as a housekeeping gene on the basis of the microarray analysis that showed its expression is unchanged following treatment of animals with pSMAA-hSlo or pVAX-hSlo. As described above, the expression level of the Slo gene is not significantly changed (Table 8). The other genes evaluated are shown in Table 8. Overall, the changes in expression determined by quantitative-RT-PCR support the changes in expression supported by microarray. Quantitative differences in the fold-change are likely due to the different methods of normalization used in quantitative-RT-PCR and microarray.

TABLE 8
Quantitative RT-PCR analysis of gene expression changes following
intracorporal injection of pVAX-hSlo or pSMAA-hSlo

| | pVAX-hSlo treated animals | pSMAA-hSlo treated animals |
|---|---|---|
| | (Fold change in | (Fold change in |
| | expression compared to | expression compared to |
| | pVAX treated animals) | pVAX treated animals) |
| Vcsa1 | 10.56 ± 2.43 | 320 ± 82 |
| Slo | 1.23 ± 0.51 | 1.33 ± 0.61 |
| Muc10 | 17.425 ± 2.3 | 210.5 ± 142.5 |
| Alas2 | 19 ± 3.23 | 5.84 ± 1.34 |
| Pbsn | 4.7 ± 1.16 | 2.16 ± 1.05 |
| S100a9 | 6.46 ± 1.62 | 4.6 ± 1.875 |
| Krt1-18 | 1.56 ± 0.06 | 2.03 ± 0.23 |
| Expi | 11.16 ± 2.62 | 42.53 ± 11.23 |

RNA was extracted from three groups of 5 rats each, treated with pVAX-hSlo, pSMAA-hSlo or pVAX as control and analyzed by quantitative RT-PCR. Results are the average fold change in expression (± distribution) compared to control.

The expression of genes changed in response to intracorporal injection of pVAX-hSlo or pSMAA-hSlo were determined in corporal smooth muscle cells transfected with pVAX-hSlo or pSMAA-hSlo in vitro. The changes in gene expression observed one week following intracorporal gene transfer of pSMAA-hSlo or pVAX-hSlo may have been a result of the physiological improvement of erectile function, rather than a direct consequence of gene transfer of the plasmids expressing hSlo. In order to test this, cultured rat smooth muscle corporal cells were transfected in vitro with pVAX-hSlo or pSMAA-hSlo plasmids, and gene expression was compared with control cells (transfected with pVAX). Forty eight hours after transfection, cells were harvested, RNA was extracted, and gene expression levels were determined by quantitative-RT-PCR. In contrast to the levels of Slo detected in corporal tissue, in vitro transfection of cells with plasmids expressing hSlo resulted in significantly higher expression of the Slo gene. Slo expression following transfection with pVAX-hSlo was elevated >14-fold and >100,000 fold with pSMAA-hSlo. The greater expression of the Slo gene from pSMAA-hSlo may be facilitated by greater efficiency of the smooth muscle alpha actin promoter, as previously observed (Melman et al. 2008).

Genes changed in expression when corporal cells were transfected with pVAX-hSlo, pSMAA-hSlo or pVAX were analyzed using quantitative-RT-PCR, focusing on genes that were among the most changed in expression in corporal tissue after the in vivo administration of the plasmids, either up-regulated (Vcsa1, EXPI, KRT1-18,) or down-regulated (Cav, RGD:1303126, EMP1, Eefla1). None of the genes investigated were significantly changed in expression.

Microarray analysis was performed on the cells transfected with pVAX-hSlo and gene expression compared to cells treated with the empty plasmid backbone (pVAX). A total of a 166 genes was changed in response to over-expression of hSlo (31 genes up-regulated, 136 down regulated). None of these genes corresponded to genes changed in expression in the corpora of animals treated with pVAX-hSlo.

Administration of tadalafil and pVAX-hSlo results in up-regulation of Vcsa1. Experiments were conducted to determine if the expression of Vcsa1 would correlate with the recovery of erectile function not only following gene transfer treatments but also following the administration of a PDE5 inhibitor (tadalafil) alone, or in association with pVAX-hSlo in retired breeder rats.

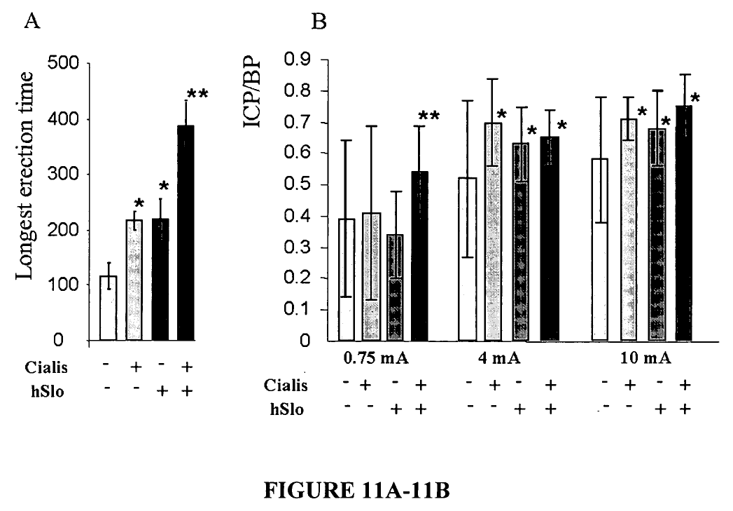

Recovery of erectile function was evaluated by measuring the longest visually observed erection (FIG. 11A) and by ICP/BP determination (FIG. 11B). For rats treated either with tadalafil or with pVAX-hSlo, the longest measured erection time was approximately 210 seconds, about 100 seconds longer than the untreated control groups, while a combination of the two treatments led to almost a two-fold increase in the length of erection. The ICP/BP response was measured following electrostimulation of a cavernous nerve with 0.4, 4 and 10 mA. All treatments produced a significant improvement in the erectile capacity at 4 and 10 mA stimulation compared to control groups. The combinatorial treatment (gene transfer of pVAX-hSlo and tadalafil) showed a slight improvement in the ICP/BP ratio over the single treatments alone particularly at 0.75 mA of electrostimulation.

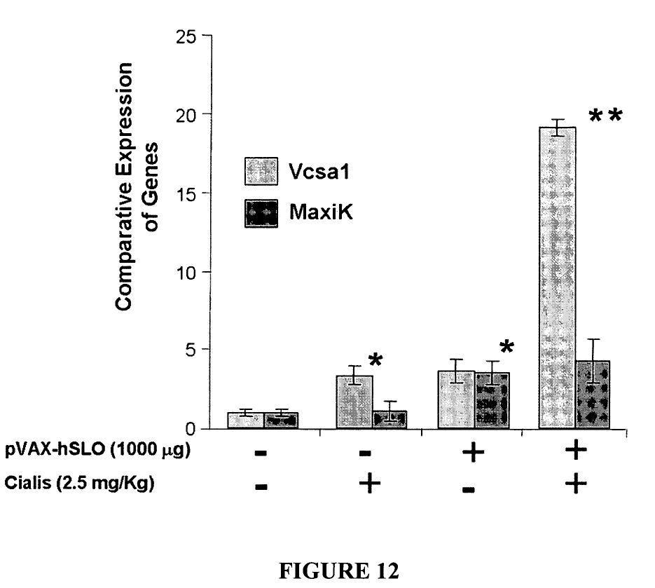

Recovery of erection is associated with increase of Vcsa1 expression. Quantitative-RT-PCR was used to analyze the expression of the Vcsa1 and hSlo transcripts after administration of tadalafil, pVAX-hSlo or a combination of the two treatments that restore erectile function. Results indicate that intracorporal gene transfer of pVAX-hSlo results in higher (but not statistically, p>0.05) levels of the hSlo transcript after 4 weeks (FIG. 12). To verify if recovery of erection through different treatments correlates with findings showing up-regulation of Vcsa1 subsequent to gene transfer of plasmids expressing hSlo, quantitative RT-PCT was performed to determine the expression level of Vcsa1. Interestingly, Vcsa1 increased in expression by approximately 4-fold (FIG. 11). Tadalafil, even though administered only 2 hours prior to the measurement of erectile function and subsequent harvesting of the tissue for RNA extraction, also up-regulates Vcsa1 expression by approximately 4-fold. This indicates that the expression of the Vcsa1 gene is rapidly activated when erectile function is recovered by administration of PDE5 inhibitors. Interestingly, the combination therapy seems to have a synergistic effect on the level of Vcsa1 expression. The detected level of Vcsa1 transcript is approximately 20-fold greater than untreated animals and five-fold greater than when the individual treatments are given. Although there is not a greater increase in the ICP/BP when the combination of treatments is used compared to the individual treatments (FIG. 11B) this may be a reflection of a plateau reached in the ICP/BP measurement following a single treatment. In an alternative measure of erectile function (longest erection time) the combination of treatments does show an effect greater than the two treatments used on their own (FIG. 11A). The level of Vcsa1 expression may therefore reflect the efficacy of ED treatment.

### Discussion

The work presented here demonstrates that a sub-set of genes are changed in corporal tissue following gene transfer of plasmids that express hSlo and cause improved erectile function. These genes are distinct from gene changes that occur as a primary response to over-expression of hSlo. As a group, these genes represent molecular markers for erectile function, and their expression may reflect the efficacy of ED treatments rather than a direct response to the treatment itself.

The pVAX-hSlo vector has been shown to improve both erectile and bladder function in animal models (Christ et al. 1998, 2001; Melman et al. 2003), and has been evaluated in phase I clinical trials for the treatment of ED (Melman et al. 2005). A gene transfer vector (pSMAA-hSlo) in which hSlo expressed from a smooth muscle specific promoter was also shown to be effective in treating ED in aging rats (Melman et al. 2008). Gene transfer of plasmids that restore erectile function have two characteristics that make them useful for identifying the genes that are changed as a result of improved erectile function, rather than a direct effect of the expressed gene. Firstly, the pVAX-hSlo plasmid can improve erectile function in rats from one week to at least 6 months following a single intra-corporal injection, without any significant affect on the overall level of Slo in the penis (Melman et al. 2003). Secondly, only a small population of cells in the corpora actually take up the plasmid (Christ et al. 1998). These factors would reduce the likelihood of any gene changes (when looking at the entire corpora) to be a direct effect of the over-expression of hSlo.

In the present study, the ED present in retired breeder Sprague-Dawley rats was normalized by intracorporal injection of the plasmids pVAX-hSlo or pSMAA-hSlo, and erectile function and gene expression were compared to an empty plasmid vector, pVAX (the control). After confirming both plasmids expressing hSlo improve erectile function, Affymetrix Rat230_{—}2 microarrays were used to analyze changes in the global gene expression pattern in the rats' corporal tissue. A number of genes were identified that are either up- or down-regulated as a consequence of improved erectile function following administration of pVAX-hSlo or pSMAA-hSlo (compared to a pVAX treated control animal). The two plasmids expressing hSlo both caused a similar improvement of erectile function, as determined by ICP/BP, and there is considerable overlap between the lists of changed genes. The differences that exist in the lists of changed genes with the two plasmids could be a result of several factors. For example, whereas pVAX-hSlo uses a CMV promoter, which has promoter activity in a wide range of cell types, pSMAA-hSlo has a smooth muscle specific promoter. When the entire corporal tissue (containing nerves, endothelium, fibrous tissue, as well as smooth muscle) is analyzed for gene expression the targeting of a specific cell type (i.e., only smooth muscle) may cause a different spectrum of genes to be activated. The plasmid pSMAA-hSlo appears more efficient at expressing the hSlo gene. The locally higher concentrations of the hSlo transcript could potentially also cause differences between the animals treated with pSMAA-hSlo and pVAX-hSlo. Also, the use of 4 microarrays per group may not be sufficient to completely eliminate inter-animal variation and experimental differences.

Ontological analysis showed that intermediate filaments are up-regulated in animals treated with both pVAX-hSlo and pSMAA-hSlo. These filaments form a distinct elongated structure that occurs in the cytoplasm of eukaryotic cells and are involved in mechanically integrating the various components of the cytoplasmic space. They are important regulators of smooth muscle tone, and therefore their over-expression with treatments that improve erectile function could be physiologically relevant (Tang 2008). Genes involved in transcriptional regulation are over-represented amongst the group of down-regulated genes. The change in the expression of these genes may represent an adaptive response to the normalization of erectile function.

Several of the most up-regulated genes in corpora treated with both plasmids such as Vcsa1, RGD: 708577 and Muc10 are associated with expression in the submandibular gland. It is possible that transcription of these genes is linked in a common transcriptional unit. Muc10 and Vcsa1 (but not RGD: 708577) are located on consecutive positions on chromosome 14, respectively 14p21 and 14p22-p21.

Interestingly, none of the reported genes changed in expression studies performed to date following the treatment of ED using PDE5 inhibitors (Andrade et al. 2008, Jung et al. 2007) overlap with the genes changed in the present study. This could be a result of studies done in different models of ED. However, at least one of the genes identified herein as changed in expression following treatment of the aging model of ED (Vcsa1) was also changed in models of ED (aging, neurogenic and diabetes). It is likely the design of the previously reported studies detected changes in gene expression as a direct consequence of the treatment, rather than changes reflecting improvement of erectile function.

Both microarray and RT-PCR analysis show that the overall expression level of MaxiK (the transcript of the Slo gene) is not significantly increased. It has previously been demonstrated that relatively few cells actually take up intracorporally injected plasmid, and overall the level of the Slo gene expression in the penis is not significantly affected when either pVAX-hSlo or pSMAA-hSlo is administered to animals despite a normalization of erectile function (Christ et al. 1998, Melman et al. 2003, 2008). Since only a small population of cells take up the plasmid, these cells may express locally high levels of hSlo, and then influence their neighboring cells, possibly through a gap-junction network.

The variable coding sequence protein Al, Vcsa1, was among the most up-regulated genes. Vcsa1 has been indicated to be a marker of ED in several animal models. In addition, human homologues of Vcsa1 (ProL1 and hSMR3A/B) are down-regulated in the corpora of human patients with ED. In order to confirm that Vcsa1 expression responded to other types of treatments for ED, ED was treated in retired breeder with a PDE5 inhibitor, tadalafil. Pharmacotherapy also resulted in a significant increase in Vcsa1 expression, remarkably when it is considered that tadalafil was only administered approximately 2 hours before the physiological/molecular determinations. Furthermore, these experiments show that there is a correlation between the level of recovery of erection and the amount of Vcsa1 up-regulation. A combinatorial treatment of tadalafil and pVAX-hSlo demonstrated a significant increase in the longest observed erection time, which corresponded to a 5-fold up-regulation of Vcsa1 than in either experiment when the treatments were applied separately.

In conclusion, Vcsa1, is up-regulated in response to both gene- and pharmaco-therapeutic treatments for ED. A combination of both treatments causes an even greater, synergistic, affect on Vcsa1 expression, suggesting that it could potentially act as a quantitative measure of the efficacy of ED treatments. Previous results have shown that in patients, the human homologues of Vcsa1, (hSMR3 and ProL1) are markers of ED. The identification of an easily assayable objective marker for erectile function in humans is of great value in the evaluation of the efficacy of treatments for ED.

### Example VI

### Antibody for hSMR3A Protein

A peptide sequence from hSMR3A/B was synthesized (TPGESQRGPRGPYP) (SEQ ID NO:24) and used to generate a polyclonal antibody in rabbits. This sequence was chosen because of its high antigen potential, no homology to existing sequences in GenBank, and because it contains closest homology to the region in Vcsa1 that generates the sialorphin peptide (QHNPR) (SEQ ID NO:25). A commercial source (GenScript, NJ) was used to synthesize the peptide and generate a rabbit polyclonal antibody.

The antibody for hSMR3 was demonstrated to detect a band corresponding to the gene product of hSMR3A by transfecting CSM cells with pVAX-Vcsa1 and performing a Western blot analysis. As a control, cells were transfected with the empty vector. A protein band of 26 kD was observed. This is larger that the 14 kD protein product expected from the amino acid sequence, but corresponds with the reported size of the rat homologue (Vcsa1) reported by User et al (2003).

The antibody for hSMR3 has been used to develop a radioimmunoassay (RIA) for detecting SMR3 in human patients and following intracorporal injection of pVAX-hSMR3A in rats. The detection limit is as low as 1 ng, which is in the range that the rat homologue (sialorphin) is found in the plasma of rats. Initial studies on humans have looked at the level of SMR3 in the serum of 5 diabetic patients, and 2 (non-diabetic) controls. SMR3 was also detectable in the saliva of 5 (non-diabetic) control patient samples. Given that obtaining saliva for analysis is non-invasive, saliva may be the best choice for performing the analysis of SMR3 in patients.

### REFERENCES

Andersson, K. E., Pharmacology of penile erection. Pharmacol Rev, 2001. 53(3): p. 417-50.
Andersson, K. E., Erectile physiological and pathophysiological pathways involved in erectile dysfunction. J Urol, 2003. 170(2 Pt 2): p. S6-13; discussion S13-4.
Andrade E, Andrade P M, Borra R C, Claro J, and Srougi M. cDNA microarray analysis of differentially expressed genes in penile tissue after treatment with tadalafil. BJU Int 101: 508-512, 2008.
Bleustein C B, Arezzo J C, Eckholdt H, Melman A. The neuropathy of erectile dysfunction. Int J Impot Res 2002: 14:433-9.
Burnett, A. L., Erectile dysfunction. J Urol, 2006. 175(3 Pt 2): p. S25-31.
Christ G J, Day N S, Day M, Santizo C, Zhao W, Sclafani T, Zinman J, Hsieh K, Venkateswarlu K, Valcic M, and Melman A. Bladder injection of “naked” hSlo/pcDNA3 ameliorates detrusor hyperactivity in obstructed rats in vivo. Am J Physiol Regul Integr Comp Physiol 281: R1699-1709, 2001.
Christ G J, Day N, Santizo C, Sato Y, Zhao W, Sclafani T, et al. Intracorporal injection of hSlo cDNA restores erectile capacity in STZ-diabetic F-344 rats in vivo. Am J Physiol Heart Circ Physiol 2004: 287:H1544-53.
Christ G J, Rehman J, Day N, Salkoff L, Valcic M, Melman A, et al. Intracorporal injection of hSlo cDNA in rats produces physiologically relevant alterations in penile function. Am J Physiol 1998: 275:H600-8.
Corpet, F. Multiple sequence alignment with hierarchical clustering, Nucleic Acids Res 16 (1988), p. 10881-90.
Courty Y, Rosinski-Chupin I, Rougeon F: A new proline-rich protein precursor expressed in the salivary glands of the rat is encoded by a gene homologous to the gene coding for the prohormone-like protein SMR1. J Biol Chem 1994, 269:520-27.
Courty Y, Rosinski-Chupin I, Rougeon F: Various transcripts are generated from the VCSA1 gene by alternative splicing and poly(A) processing in the rat submandibular gland. Gene 1995, 162:291-96.
Courty Y, Singer M, Rosinski-Chupin I, Rougeon F: Episodic evolution and rapid divergence of members of the rat multigene family encoding the salivary prohormone-like protein SMR1. Mol Biol Evol 1996, 13:758-66.
Dickinson D P, Thiesse M: cDNA cloning of an abundant human lacrimal gland mRNA encoding a novel tear protein. Curr Eye Res 1996, 15:377-86.
Eaton, C. B., et al., A retrospective study of the relationship between biomarkers of atherosclerosis and erectile dysfunction in 988 men. Int J Impot Res. 2007 March-April; 19(2):218-25. Epub 2006 Aug. 17.
Feldman, H. A., et al., Impotence and its medical and psychosocial correlates: results of the Massachusetts Male Aging Study. J Urol, 1994. 151(1): p. 54-61.
Grover, S. A., et al., The prevalence of erectile dysfunction in the primary care setting: importance of risk factors for diabetes and vascular disease. Arch Intern Med, 2006. 166(2): p. 213-9.
Hai, C. M. and R. A. Murphy, Regulation of shortening velocity by cross-bridge phosphorylation in smooth muscle. Am J Physiol, 1988. 255(1 Pt 1): p. C86-94.
Hartshorne, D. J., M. Ito, and F. Erdodi, Myosin light chain phosphatase: subunit composition, interactions and regulation. J Muscle Res Cell Motil, 1998. 19(4): p. 325-41.
Isemura S: Nucleotide sequence of gene PBII encoding salivary proline-rich protein P-B. J Biochem (Tokyo) 2000, 127:393-98.
Isemura, S. and E. Saitoh, Molecular cloning and sequence analysis of cDNA coding for the precursor of the human salivary proline-rich peptide P-B, J Biochem (Tokyo) 115 (1994), p. 1101-6.
Isemura S, Saitoh E. Nucleotide sequence of gene PBI encoding a protein homologous to salivary proline-rich protein P-B. J Biochem (Tokyo) 1997: 121:1025-30.
Isemura, S., S. Watanabe, S. Fujiwara and K. Sanada, Tissue distribution and nucleotide sequence of bovine mRNA for salivary proline-rich protein P-B, Arch Oral Biol 49 (2004), p. 881-7.
Jackson W F, Huebner J M, and Rusch N J. Enzymatic isolation and characterization of single vascular smooth muscle cells from cremasteric arterioles. Microcirculation 3: 313-328, 1996.
Jung H G, Shin J H, Kim K W, Yu J Y, Kang K K, Ahn B O, Kwon J W, and Yoo M. Microarray analysis of gene expression profile in the corpus cavernosum of hypercholesterolemic rats after chronic treatment with PDE5 inhibitor. Life Sci 80: 699-708, 2007.
Kawasaki K, Suzuki T, Weiss K M: Genetic basis for the evolution of vertebrate mineralized tissue. Proc Natl Acad Sci USA 2004, 101:11356-61.
Kawasaki K, Weiss K M: Mineralized tissue and vertebrate evolution: the secretory calcium-binding phosphoprotein gene cluster. Proc Natl Acad Sci USA 2003, 100:4060-65.
Kawasaki K, Weiss K M: Evolutionary genetics of vertebrate tissue mineralization: the origin and evolution of the secretory calcium-binding phosphoprotein family. J Exp Zoolog B Mol Dev Evol 2006, 306:295-316.
Kim S Z, Kim S H, Park J K, Koh G Y, Cho K W. Presence and biological activity of C-type natriuretic peptide-dependent guanylate cyclase-coupled receptor in the penile corpus cavernosum. J Urol 1998; 159(5): 1741-6.
Kirby, M., G. Jackson, and U. Simonsen, Endothelial dysfunction links erectile dysfunction to heart disease. Int J Clin Pract, 2005. 59(2): p. 225-9.
Korenman, S. G. Epidemiology of erectile dysfunction, Endocrine 23 (2004), p. 87-91.
Kupelian, V., et al., Erectile dysfunction as a predictor of the metabolic syndrome in aging men: results from the Massachusetts Male Aging Study. J Urol, 2006. 176(1): p. 222-6.
Kuthe A, Reinecke M, Uckert S, Becker A, David I, Heitland A, et al. Expression of guanylyl cyclase B in the human corpus cavernosum penis and the possible involvement of its ligand C-type natriuretic polypeptide in the induction of penile erection. J Urol 2003; 169(5):1918-22.
Livak K J, Schmittgen T D. Analysis of relative gene expression data using real-time quantitative PCR and the 2(-Delta Delta C(T)) Method. Methods 2001: 25:402-8.
Lizza, E. F. and R. C. Rosen, Definition and classification of erectile dysfunction: report of the Nomenclature Committee of the International Society of Impotence Research, Int J Impot Res 11 (1999), p. 141-3.
Marshall, S. M. and A. Flyvbjerg, Prevention and early detection of vascular complications of diabetes. BMJ, 2006. 333(7566): p. 475-80.
Marton Z, Pataricza J, Krassoi I, Varro A, Papp J G. NEP inhibitors enhance C-type natriuretic peptide-induced relaxation in porcine isolated coronary artery. Vascul Pharmacol 2005; 43(4):207-12.
Melman, A., et al., The first human trial for gene transfer therapy for the treatment of erectile dysfunction: preliminary results. Eur Urol, 2005. 48(2): p. 314-8.
Melman A, Biggs G, Davies K, Zhao W, Tar M T, and Christ G J. Gene transfer with a vector expressing Maxi-K from a smooth muscle-specific promoter restores erectile function in the aging rat. Gene Ther 15: 364-370, 2008.
Melman, A., J. Fogarty and J. Hafron, Can self-administered questionnaires supplant objective testing of erectile function? A comparison between the International Index of Erectile Function and objective studies, Int J Impot Res 18 (2006), p. 126-129.
Melman A, Zhao W, Davies K P, Bakal R, Christ G J. The successful long-term treatment of age related erectile dysfunction with hSlo cDNA in rats in vivo. J Urol 2003: 170:285-90.
Messaoudi, M., D. Desor, A. Nejdi and C. Rougeot, The endogenous androgen-regulated sialorphin modulates male rat sexual behavior, Horm Behav 46 (2004), p. 684-91.
Moore, R. A., S. Derry, and H. J. McQuay, Indirect comparison of interventions using published randomised trials: systematic review of PDE-5 inhibitors for erectile dysfunction. BMC Urol, 2005.5: p. 18.
Muller, A. and J. P. Mulhall, Cardiovascular disease, metabolic syndrome and erectile dysfunction, Curr Opin Urol 16 (2006), p. 435-43.
Rijnkels M, Elnitski L, Miller W, Rosen J M: Multispecies comparative analysis of a mammalian-specific genomic domain encoding secretory proteins. Genomics 2003, 82:417-32.
Rosinski-Chupin, I., J. F. Huaulme, C. Rougeot and F. Rougeon, The transcriptional response to androgens of the rat VCSA1 gene is amplified by both binary and graded mechanisms, Endocrinology 142 (2001), p. 4550-9.
Rosinski-Chupin I, Kuramoto T, Courty Y, Rougeon F, Serikawa T: Assignment of the rat variable coding sequence (VCS) gene family to chromosome 14. Mamm Genome 1995, 6:153-54.
Rosinski-Chupin I, Rougeon F. The gene encoding SMR1, a precursor-like polypeptide of the male rat submaxillary gland, has the same organization as the preprothyrotropin-releasing hormone gene. DNA Cell Biol 1990, 9:553-59.
Rosinski-Chupin I, Rougeot C, Courty Y, Rougeon F. Localization of mRNAs of two androgen-dependent proteins, SMR1 and SMR2, by in situ hybridization reveals sexual differences in acinar cells of rat submandibular gland. J Histochem Cytochem 1993: 41:1645-9.
Rosinski-Chupin, I., D. Tronik and F. Rougeon, High level of accumulation of a mRNA coding for a precursor-like protein in the submaxillary gland of male rats, Proc Natl Acad Sci USA 85 (1988), p. 8553-7.
Rougeot, C., M. Messaoudi, V. Hermitte, A. G. Rigault, T. Blisnick and C. Dugave et al., Sialorphin, a natural inhibitor of rat membrane-bound neutral endopeptidase that displays analgesic activity, Proc Natl Acad Sci USA 100 (2003), p. 8549-54.
Rougeot, C., I. Rosinski-Chupin, E. Njamkepo and F. Rougeon, Selective processing of submandibular rat 1 protein at dibasic cleavage sites Salivary and bloodstream secretion products, Eur J Biochem 219 (1994), p. 765-73.
Rougeot C, Rosinski-Chupin I, Rougeon F: Novel genes and hormones in salivary glands: From the gene for the submandibular rat1 protein (SMR1) precursor to receptor sites for SMR1 mature peptides. Biomedical Reviews 1998, 9:17-32.
Rougeot C, Vienet R, Cardona A, Le Doledec L, Grognet J M, Rougeon F. Targets for SMR1-pentapeptide suggest a link between the circulating peptide and mineral transport. Am J Physiol 1997: 273:R1309-20.
Sambrook J and Russell D W. Molecular Cloning. A Laboratory Manual, third edition, Cold Spring Harbor Laboratory Press, 2001.
Samuel, M., et al., Effects of phosphorylation, magnesium, and filament assembly on actin-activated, ATPase of pig urinary bladder myosin. Biochemistry, 1990. 29(30): p. 7124-32.
Sensorale-Pose M, Rougeon F: The mouse Vcs2 gene is a composite structure which evolved by gene fusion and encodes five distinct salivary mRNA species. Gene 1997, 201:75-85.
Shabsigh, R. and A. G. Anastasiadis, Erectile dysfunction. Annu Rev Med, 2003. 54: p. 153-68.
Shabsigh, R., M. A. Perelman, D. C. Lockhart, T. F. Lue and G. A. Broderick, Health issues of men: prevalence and correlates of erectile dysfunction, J Urol 174 (2005), p. 662-7.
Sullivan, C. J., T. H. Teal, I. P. Luttrell, K. B. Tran, M. A. Peters and H. Wessells, Microarray analysis reveals novel gene expression changes associated with erectile dysfunction in diabetic rats, Physiol Genomics 23 (2005), p. 192-205.
Stuckey, B. G., et al., Erectile dysfunction predicts generalised cardiovascular disease: Evidence from a case-control study. Atherosclerosis, 2006.
Tang D D. Intermediate filaments in smooth muscle. Am J Physiol Cell Physiol 294: C869-878, 2008.
Thompson, I. M., C. M. Tangen, P. J. Goodman, J. L. Probstfield, C. M. Moinpour and C. A. Coltman, Erectile dysfunction and subsequent cardiovascular disease, JAMA 294 (2005), p. 2996-3002.
Tronik-Le Roux D, Sensorale-Pose M, Rougeon F: Three novel SMR1-related cDNAs characterized in the submaxillary gland of mice show extensive evolutionary divergence in the protein coding region. Gene 1994, 142:175-82.
User, H. M. D. J. Zelner, K. E. McKenna and K. T. McVary, Microarray analysis and description of SMR1 gene in rat penis in a post-radical prostatectomy model of erectile dysfunction, J Urol 170 (2003), p. 298-301.
Walther T, Stepan H. C-type natriuretic peptide in reproduction, pregnancy and fetal development. J Endocrinol 2004; 180(1):17-22.
Wisner, A., E. Dufour, M. Messaoudi, A. Nejdi, A. Marcel and M. N. Ungeheuer et al., Human opiorphin, a natural antinociceptive modulator of opioid-dependent pathways, Proc Natl Acad Sci USA 103 (2006), p. 17979-84.

1. A method for determining whether a subject has erectile dysfunction, bladder dysfunction, cardiovascular disease or hypertension, the method comprising determining the expression of a human Vcsa1 family member in the subject, wherein decreased expression of the human Vcsa1 family member is indicative of erectile dysfunction, bladder dysfunction, cardiovascular disease or hypertension in the subject.

2. A method for monitoring the efficacy of treatment of a subject for erectile dysfunction, bladder dysfunction, cardiovascular disease or hypertension, the method comprising determining the expression of a human Vcsa1 family member in a subject undergoing treatment for erectile dysfunction, bladder dysfunction, cardiovascular disease or hypertension, wherein increased expression of the human Vcsa1 family member is indicative of effective treatment or wherein decreased expression of the human Vcsa1 family member is indicative of a need to continue treatment.

3. The method of claim 1, wherein the amount of decreased expression of the human Vcsa1 family member is indicative of the degree of pathology of erectile dysfunction, bladder dysfunction, cardiovascular disease or hypertension.

4. The method of claim 2, wherein the amount of increased expression of the human Vcsa1 family member is indicative of the degree of the effectiveness of treatment of erectile dysfunction, bladder dysfunction, cardiovascular disease or hypertension.

5. The method of claim 1, wherein the subject has erectile dysfunction.

6. The method of claim 1, wherein the subject has bladder dysfunction.

7. The method of claim 1, wherein the subject has hypertension.

8. The method of claim 1, wherein the subject has cardiovascular disease.

9. The method of claim 5, wherein the erectile dysfunction is organic erectile dysfunction.

10. The method of claim 9, wherein the erectile dysfunction is associated with diabetes and/or aging.

11. The method of claim 5, wherein the method distinguishes organic erectile dysfunction from psychogenic erectile dysfunction, where a decreased level of expression of the human Vcsa1 family member is indicative of organic erectile dysfunction and a normal level of expression of the human Vcsa1 family member is indicative of psychogenic erectile dysfunction.

12. The method of claim 1, wherein the human Vcsa1 family member is obtained from a blood, saliva or corpus cavernosum tissue sample from the subject.

13. The method of claim 1, wherein the human Vcsa1 family member is hSMR3A, hSMR3B or PROL1.

14. The method of claim 13, wherein hSMR3A has the nucleotide sequence set forth in SEQ ID NO:15.

15. The method of claim 13, wherein hSMR3B has the nucleotide sequence set forth in SEQ ID NO:16.

16. The method of claim 13, wherein PROL1 has the nucleotide sequence set forth in SEQ ID NO:17.

17. The method of claim 1, wherein expression of the human Vcsa1 family member is determined by measuring mRNA expression.

18. The method of claim 17, wherein the human Vcsa1 family member is hSMR3A, hSMR3B or PROL1.

19. The method of claim 1, wherein expression of the human Vcsa1 family member is determined by measuring expression of a protein or a peptide portion of the protein.

20. The method of claim 19, wherein the human Vcsa1 family member is hSMR3A, hSMR3B or PROL1.

21. The method of claim 20, wherein the protein is hSMR3A (SEQ ID NO:19).

22. The method of claim 20, wherein the protein is hSMR3B (SEQ ID NO:20).

23. The method of claim 20, wherein the protein is PROL1 (SEQ ID NO:21).

24. The method of claim 19, wherein the peptide is opiorphin (QRFSR) (SEQ ID NO:23).


SEQUENCE LISTING (48 sequences)

SEQ ID NO: 1 - DNA, 22 bp, Artificial Sequence
  forward primer for pVAX-Vcsa1
caaggggctaccaaagatgaag

SEQ ID NO: 2 - DNA, 22 bp, Artificial Sequence
  reverse primer for pVAX-Vcsa1
ccaaaaggaatttattatttgc

SEQ ID NO: 3 - DNA, 17 bp, Artificial Sequence
  forward primer for Vcsa1
gagggtgtcagaggccc

SEQ ID NO: 4 - DNA, 22 bp, Artificial Sequence
  reverse primer for Vcsa1
gagcagttagctgccactgata

SEQ ID NO: 5 - DNA, 22 bp, Artificial Sequence
  forward primer for GAPDH
gccgcctgcttcaccaccttct

SEQ ID NO: 6 - DNA, 23 bp, Artificial Sequence
  reverse primer for GAPDH
gcatggccttccgtgttcctacc

SEQ ID NO: 7 - DNA, 23 bp, Artificial Sequence
  SMR3AF primer
ggatgaaatcactgacttggatc

SEQ ID NO: 8 - DNA, 23 bp, Artificial Sequence
  SMR3AR primer
gtatttagggtgcaggagtaggg

SEQ ID NO: 9 - DNA, 21 bp, Artificial Sequence
  hSMR3A forward primer
ctatggtccagggagatttcc

SEQ ID NO: 10 - DNA, 22 bp, Artificial Sequence
  hSMR3A reverse primer
gaggaggaagagagtgtgattg

SEQ ID NO: 11 - DNA, 26 bp, Artificial Sequence
  Prol1 forward primer
gctcttatttcatgtttcacacccag

SEQ ID NO: 12 - DNA, 21 bp, Artificial Sequence
  Prol1 reverse primer
taacccggaaagagtcgaggt

SEQ ID NO: 13 - DNA, 26 bp, Artificial Sequence
  Slo forward primer
tacttcaatgacaatatcctcaccct

SEQ ID NO: 14 - DNA, 25 bp, Artificial Sequence
  Slo reverse primer
accataacaaccaccatcccctaag

SEQ ID NO: 15 - DNA, 405 bp, Human
atgaaatcactgacttggatcttgggcctttgggctcttgcagcgtgtttcacacctggtgagagtcaaagaggccccaggggaccatatccacctggaccactggctcctcctcctccaccatgttttccttttggaacaggatttgttccaccaccccatcctccaccctatggtccagggagatttccaccacccctttctccaccctatggtccagggagaatcccaccatcccctcctccaccctatggtccagggagaattcaatcacactctcttcctcctccttatggcccaggttatccacagccaccttcccaaccaagaccctatccacctggacctccatttttccctgtaaattctccaactgatcctgccctccctactcctgcaccctaa

SEQ ID NO: 16 - DNA, 240 bp, Human
atgaaatcactgacttggatcttgggcctttgggctcttgcagcgtgtttcacacctggtgagagtcaaagaggccccaggggaccatatccacctggaccgctggctcctcctcaaccttttggcccaggatttgttccaccacctcctcctccaccctatggtccagggagaatcccacctcctcctcccgcaccctatggtccagggatatttccaccaccccctcctcaaccctaa

SEQ ID NO: 17 - DNA, 746 bp, Human
atgaaattaactttcttcttgggcctgttggctcttatttcatgtttcacacccagtgagagtcaaagattctccagaagaccatatctacctggccagctgccaccacctccactctacaggccaagatgggttccaccaagtcccccacctccctatgactcaagacttaattcaccactttctcttccctttgtcccagggcgagttccaccatcttctttctctcgatttagccaagcagtcattctatctcaactctttccattggaatctattagacaacctcgactctttccgggttatccaaacctacatttcccactaagaccttactatgtaggacctattaggatattaaaacccccatttcctcctattcctttttttcttgctatttaccttcctatctctaaccctgagccccaaataaacatcaccaccgcagatacaacaatcaccacaaatccccccaccactgcaacagcaaccaccagcacttccacaaaacccacaatgacgatcagctcctcaacagtacctatctcttcaacaccagagcctgccacctccatatcagcagcaacccccgcagcatctactgaaaatactactcaaattctcgccaaccgtcctcacacagtattgctcaatgccactgtccaagttacgacttccaaccaaactatattaagcagcccagcctttaaaagtttttggcaaaaactctttgccatttttggtta

SEQ ID NO: 18 - DNA, 441 bp, Rat
atgaagtcactgtatttgatctttggcctgtggatccttctagcatgcttccagtcaggtgagggtgtcagaggcccaagaagacaacataatcctagaagacaacaagatccttcaactcttcctcattatcttggtcttcagcctgatcccaatggtggacaaataggagtaacaatcactatacccttaaatcttcaaccacctcgtgttcttgttaatcttcccggttttatcactggaccaccattggttgtacaaggtaccactgaatatcaatatcagtggcagctaactgctccagaccctacacctctaagcaatcctcctactcaacttctttccacagaacaagcaaatacaaaaacagatgccaaaatctccaacactactgcgactacccaaaattccactgatatttttgaaggtggtggcaaataa

SEQ ID NO: 19 - protein, 134 aa, Human
MKSLTWILGLWALAACFTPGESQRGPRGPYPPGPLAPPPPPRFPFGTGFVPPPHPPPYGPGRFPPPLSPPYGPGRIPPSPPPPYGPGRIQSHSLPPPYGPGYPQPPSQPRPYPPGPPFFPVNSPTDPALPTLAP

SEQ ID NO: 20 - protein, 106 aa, Human
MKSLTWILGLWALAACFTPGESQRGPRGPYPPGPLAPPQPFGPGFVPPPPPPPYGPGRIPPPYSCTPNMNNCSRCHHHHKRHHYPCNYCFCYPKYEFQHCFQETFT

SEQ ID NO: 21 - protein, 248 aa, Human
MKLTFFLGLLALISCFTPSESQRFSRRPYLPGQLPPPPLYRPRWVPPSPPPPYDSRLNSPLSLPFVPGRVPPSSFSRFSQAVILSQLFPLESIRQPRLFPGYPNLHFPLRPYYVGPIRILKPPFPPIPFFLAIYLPISNPEPQINITTADTTITTNPPTTATATTSTSTKPTMTISSSTVPISSTPEPATSISAATPAASTENTTQILANRPHTVLLNATVQVTTSNQTILSSPAFKSFWQKLFAIFG

SEQ ID NO: 22 - protein, 86 aa, Rat
MKSLYLIFGLWILLACFQSGEGVRGPRRQHNPRRQQDPSTLPHYLGLQPDPNGGQIGVTITKTDAKISNTTATTQNSTDIFEGGGK

SEQ ID NO: 23 - protein, 5 aa, Human Opiorphin Peptide
QRFSR

SEQ ID NO: 24 - protein, 14 aa, Human hSMR3A/B fragment
TPGESQRGPRGPYP

SEQ ID NO: 25 - protein, 5 aa, Rat sialorphin peptide
QHNPR

SEQ ID NO: 26 - protein, 7 aa, Rat SGPT Peptide
TDIFEGG

SEQ ID NO: 27 - DNA, 20 bp, Artificial Sequence
  forward primer of Expi
tgttccaatggctgtggtca

SEQ ID NO: 28 - DNA, 21 bp, Artificial Sequence
  reverse primer of Expi
ggccatcagtcgtgcttatga

SEQ ID NO: 29 - DNA, 22 bp, Artificial Sequence
  forward primer of Krt1-18
cagaccttggagattgacctgg

SEQ ID NO: 30 - DNA, 21 bp, Artificial Sequence
  reverse primer of Krt1-18
ttgctccatctgcaccctgta

SEQ ID NO: 31 - DNA, 21 bp, Artificial Sequence
  forward primer of Cav
accatcttcggcatccctatg

SEQ ID NO: 32 - DNA, 21 bp, Artificial Sequence
  reverse primer of Cav
aggaagctcttgatgcacggt

SEQ ID NO: 33 - DNA, 22 bp, Artificial Sequence
  forward primer Eef1a1
gtcagaacgcaggtgttgtgaa

SEQ ID NO: 34 - DNA, 21 bp, Artificial Sequence
  reverse primer of Eef1a1
gccggaatctacgtgtccaat

SEQ ID NO: 35 - DNA, 19 bp, Artificial Sequence
  forward primer of Emp1
tcaaagtgcatgcccacca

SEQ ID NO: 36 - DNA, 21 bp, Artificial Sequence
  reverse primer of Emp1
gcgatggaacatgtgcatctc

SEQ ID NO: 37 - DNA, 22 bp, Artificial Sequence
  forward primer of RGD1303126
tctgacggcaggtcctatgagt

SEQ ID NO: 38 - DNA, 19 bp, Artificial Sequence
  reverse primer RGD1303126
tggccagcatctttgcatc

SEQ ID NO: 39 - DNA, 22 bp, Artificial Sequence
  forward primer of Muc10
tcccaccaaggagcaacattaa

SEQ ID NO: 40 - DNA, 21 bp, Artificial Sequence
  reverse primer Muc10
ggatgtggttttggctggaag

SEQ ID NO: 41 - DNA, 22 bp, Artificial Sequence
  forward primer of Alas2
acctcccctgctgattcagaat

SEQ ID NO: 42 - DNA, 22 bp, Artificial Sequence
  reverse primer Alas2
acggtatgtgtggtcctgcttc

SEQ ID NO: 43 - DNA, 20 bp, Artificial Sequence
  forward primer S100a9
accctgaacaaggcggaatt

SEQ ID NO: 44 - DNA, 21 bp, Artificial Sequence
  reverse primer for S100a9
tttgtgtccaggtcctccatg

SEQ ID NO: 45 - DNA, 22 bp, Artificial Sequence
  forward primer of Pbsn
tgctcacactggatgtgctagg

SEQ ID NO: 46 - DNA, 22 bp, Artificial Sequence
  reverse primer Pbsn
tccacgctactggcagctaagt

SEQ ID NO: 47 - DNA, 22 bp, Artificial Sequence
  forward primer Rp124
tcgagctgtgcagttttagtgg

SEQ ID NO: 48 - DNA, 21 bp, Artificial Sequence
  reverse primer Rp124
gcggactcacatttggcatta